(19) United States
(12) Patent Application Publication
(10) Pub. No.: US 2021/0255205 A1
(43) Pub. Date: Aug. 19, 2021

(54) COMPOSITIONS AND METHODS FOR TREATING METABOLIC DISEASE

(71) Applicant: THE REGENTS OF THE UNIVERSITY OF CALIFORNIA, Oakland, CA (US)

(72) Inventors: Peter J. Tontonoz, Los Angeles, CA (US); Jaspreet S. Sandhu, Los Angeles, CA (US)

(21) Appl. No.: 17/255,827

(22) PCT Filed: Jun. 27, 2019
(86) PCT No.: PCT/US19/39473

§ 371 Date: Dec. 23, 2020

Related U.S. Application Data
(60) Provisional application No. 62/690,510, filed on Jun. 27, 2018.

(51) Int. Cl. G01N 33/68; A61K 45/06; A61K 31/455; A61K 31/22; A61K 31/405; A61K 31/352; A61K 31/416

(52) U.S. Cl. CPC G01N 33/6896; A61K 45/06; A61K 31/455; A61K 31/416; A61K 31/405; A61K 31/352; A61K 31/22

(57) ABSTRACT

The present disclosure relates to compositions and methods for modulating a subject's cholesterol levels and/or treating disorders related to high cholesterol.

### RELATED APPLICATIONS

This application claims the benefit of priority to U.S. Provisional Patent Application having Ser. No. 62/690,510, filed Jun. 27, 2018, the content of which is hereby incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 26, 2019, is named UCH-14825_SL.txt and is 48,786 bytes in size.

### BACKGROUND

HDL cholesterol levels in the plasma are inversely associated with coronary heart disease (CHD) risk, but recent genetic studies have strongly implied that HDL cholesterol levels are not causally related to risk for CHD. For example, the P376L mutation in scavenger receptor class B member 1 (SR-BI) is associated with greater cardiovascular disease risk despite high plasma levels of HDL cholesterol. Mounting evidence suggests that the function of HDL, in particular its role in cholesterol movement in cells and tissues, may be key to its role in physiology and disease. HDL appears to be important in the reverse cholesterol transport pathway that brings surplus cholesterol from peripheral tissues to the liver for excretion. A better understanding of the pathways by which HDL cholesterol moves through cells and tissues may aid in the development of novel diagnostic tools and therapies.

Integral membrane receptors involved in cholesterol uptake at the plasma membrane (PM) have been characterized extensively, but little is known about mechanisms that traffic cholesterol from the plasma membrane to other compartments within the cell. Scavenger Receptor class B member 1 (SR-BI) is the principal cell-surface receptor for HDL. SR-BI is abundant in steroidogenic organs and the liver, where it facilitates the selective uptake of cholesterol (both esterified and unesterified) from HDL. In the liver, HDL-cholesterol uptake facilitates reverse cholesterol transport by delivering surplus peripheral cholesterol to hepatocytes for secretion into bile or for conversion into bile acids. In steroidogenic organs, HDL-derived cholesterol accumulates in the form of cholesterol ester, and these stores are used for the synthesis of steroid hormones. The selective HDL-cholesterol uptake pathway mediated by SR-BI is distinct from the LDLR pathway by which LDL particles are taken up and delivered to lysosomes for degradation. HDL-cholesterol uptake does not require clathrin-dependent receptor-mediated uptake or lysosomal targeting, but what happens to HDL cholesterol after SR-BI-mediated uptake is unknown. The pathways downstream of SR-BI that move cholesterol within the cell have never been defined. Accordingly, there is a great need to identify the pathways responsible for cholesterol trafficking for diagnosing and treating conditions associated with high cholesterol.

### SUMMARY OF THE INVENTION

The present invention is based, at least in part, on the characterization of the three mammalian proteins (Aster-A, -B, and -C) that bind cholesterol and facilitate its removal from the plasma membrane are described herein. The crystal structure of the central domain of Aster-A broadly resembles the sterol-binding folds of mammalian StARD and yeast Lam proteins, but sequence differences in the Aster pocket result in a distinct mode of ligand binding. The Aster N-terminal GRAM domain binds phosphatidylserine and mediates the formation of inducible plasma membrane-ER contacts in response to cholesterol accumulation in the plasma membrane. Mice lacking Aster-B are deficient in adrenal cholesterol ester storage and steroidogenesis due to an inability to transport cholesterol from SR-BI to the ER. These findings identify a nonvesicular pathway for plasma membrane to ER sterol trafficking in mammals.

In some aspects, methods of diagnosing and treating a condition associated with high cholesterol or at risk for developing a condition associated with high cholesterol are disclosed. Such methods include (a) optionally obtaining a sample from the subject; (b) determining whether the sample from the subject has a decreased Aster protein level or activity compared to a reference level representative of a subject without the condition; and (c) if the sample has a decreased Aster protein level or activity compared to the reference level, identifying the subject as having a condition associated with high cholesterol or at risk for developing a condition associated with high cholesterol. Numerous embodiments are further provided that can be applied to any aspect of the present invention described herein. For example, in some embodiments, the methods further include (d) administering a cholesterol-lowering drug to the subject, instructing the subject to reduce cholesterol uptake from diet, and/or administering a reverse cholesterol transport activator. In some embodiments, the sample is a blood sample, a liver sample, or a steroidogenic organ sample. In certain such embodiments, the steroidogenic organ is an ovary, a testis, or an adrenal gland. In some embodiments, the reference level is the Aster protein level or activity in a normal patient. In various embodiments, the condition associated with high cholesterol is coronary heart disease, stroke, peripheral vascular disease, erectile dysfunction, diabetes, high blood pressure, cellular cholesterol overload disease, and/or a disease related to defects in brain cholesterol metabolism. In some embodiments, the cellular cholesterol overload disease is Niemann-Pick type C disease. In other embodiments, the disease related to defects in brain cholesterol metabolism is Alzheimer's disease (AD), Huntington's disease (HD), or Parkinson's disease (PD). In some embodiments, the cholesterol-lowering drug is atorvastatin, fluvastatin, lovastatin, pitavastatin, pravastatin, rosuvastatin, simvastatin, niacin, colestipol, cholestyramine, colesevelam, ezetimibe, fenofibrate, gemfibrozil, alirocumab, and/or evolocumab. In certain preferred embodiments, the cholesterol-lowering drug is an agent that increases the level or activity of Aster, e.g., by activating the Aster promoter. In other embodiments, the agent comprises an Aster polypeptide or an Aster polynucleotide. In various embodiments, the Aster is Aster-A, Aster-B, and/or Aster-C. In some embodiments, the reverse cholesterol transport activator is a liver X receptor (LXR) agonist (such as LXR-623), an activator of hepatic apoA-I (such as RVX-208, apoA-I mimetic peptides or a peroxisome proliferator-activated receptor α (PPARα) agonist (e.g., LY518764)), an inhibitor of cholesteryl ester transfer protein (CETP) (such as torcetrapib, anacetrapib, or delcetrapib), or an inhibitor of endothelial lipase (EL) (such as GSK 264220A).

In some aspects, methods of diagnosing and treating a subject having a condition associated with high cholesterol or at risk for developing a condition associated with high cholesterol are disclosed. Such methods include (a) optionally obtaining a sample from the subject; (b) analyzing the sample to detect the presence of one or more mutant Aster polynucleotide molecules, and/or one or more mutant Aster polypeptides; (c) if the subject has one or more mutant Aster polynucleotide molecules, and/or one or more mutant Aster polypeptides, identifying the subject as having a condition associated with high cholesterol or at risk for developing a condition associated with high cholesterol. Numerous embodiments are further provided that can be applied to any aspect of the present invention described herein. For example, in some embodiments, the methods further include (d) administering a cholesterol-lowering drug to the patient, instructing the subject to reduce cholesterol uptake from diet, and/or administering a reverse cholesterol transport activator. In some embodiments, the sample is a blood sample, a liver sample, or a steroidogenic organ sample. In some embodiments, the steroidogenic organ is an ovary, a testis, or an adrenal gland. In some embodiments, the condition associated with high cholesterol is coronary heart disease, stroke, peripheral vascular disease, erectile dysfunction, diabetes, high blood pressure, cellular cholesterol overload disease, and/or a disease related to defects in brain cholesterol metabolism. In some embodiments, the cellular cholesterol overload disease is Niemann-Pick type C disease. In other embodiments, the disease related to defects in brain cholesterol metabolism is Alzheimer's disease (AD), Huntington's disease (HD), or Parkinson's disease (PD). In some embodiments, the cholesterol-lowering drug is atorvastatin, fluvastatin, lovastatin, pitavastatin, pravastatin, rosuvastatin, simvastatin, niacin, colestipol, cholestyramine, colesevelam, ezetimibe, fenofibrate, gemfibrozil, alirocumab, and/or evolocumab. In certain preferred embodiments, the cholesterol-lowering drug is an agent that increases the level or activity of Aster. In some embodiments, the agent comprises an Aster polypeptide and/or an Aster polynucleotide. In various embodiments, the Aster is Aster-A, Aster-B, and/or Aster-C. In some embodiments, the reverse cholesterol transport activator is a liver X receptor (LXR) agonist (such as LXR-623), an activator of hepatic apoA-I (such as RVX-208, apoA-I mimetic peptides or a peroxisome proliferator-activated receptor α (PPARα) agonist (e.g., LY518764)), an inhibitor of cholesteryl ester transfer protein (CETP) (such as torcetrapib, anacetrapib, or delcetrapib), or an inhibitor of endothelial lipase (EL) (such as GSK 264220A).

In some aspects, methods of treating a subject having a condition associated with high cholesterol or at risk for developing a condition associated with high cholesterol are disclosed. Such methods include administering to the subject an agent that increases the level or activity of Aster. Numerous embodiments are further provided that can be applied to any aspect of the present invention described herein. For example, in some embodiments, the condition associated with high cholesterol is coronary heart disease, stroke, peripheral vascular disease, erectile dysfunction, diabetes, high blood pressure, cellular cholesterol overload disease, and/or a disease related to defects in brain cholesterol metabolism. In some embodiments, the cellular cholesterol overload disease is Niemann-Pick type C disease. In other embodiments, the disease related to defects in brain cholesterol metabolism is Alzheimer's disease (AD), Huntington's disease (HD), or Parkinson's disease (PD). In some embodiments, the agent activates the Aster promoter. In some embodiments, the agent comprises an Aster polypeptide and/or an Aster polynucleotide. In various embodiments, the Aster is Aster-A, Aster-B, and/or Aster-C.

In some aspects, methods of diagnosing and treating a subject having a condition associated with high cholesterol or at risk for developing a condition associated with high cholesterol are disclosed. Such methods include (a) optionally obtaining a sample from the subject; (b) determining whether the sample from the subject has a similar Aster protein level or activity compared to a reference level representative of a subject with the condition; (c) if the sample has a similar Aster protein level or activity compared to the reference level, identifying the subject as having a condition associated with high cholesterol or at risk for developing a condition associated with high cholesterol. Numerous embodiments are further provided that can be applied to any aspect of the present invention described herein. For example, in some embodiments, the methods further include (d) administering a cholesterol-lowering drug to the subject, instructing the subject to reduce cholesterol uptake from diet, and/or administering a reverse cholesterol transport activator. In some embodiments, the sample is a blood sample, a liver sample, or a steroidogenic organ sample (such as an ovary, a testis, or an adrenal gland). In some embodiments, the reference level is the Aster protein level or activity in a normal patient. In some embodiments, the condition associated with high cholesterol is coronary heart disease, stroke, peripheral vascular disease, erectile dysfunction, diabetes, high blood pressure, cellular cholesterol overload disease, and/or a disease related to defects in brain cholesterol metabolism. In some embodiments, the cellular cholesterol overload disease is Niemann-Pick type C disease. In other embodiments, the disease related to defects in brain cholesterol metabolism is Alzheimer's disease (AD), Huntington's disease (HD), or Parkinson's disease (PD). In some embodiments, the cholesterol-lowering drug is atorvastatin, fluvastatin, lovastatin, pitavastatin, pravastatin, rosuvastatin, simvastatin, niacin, colestipol, cholestyramine, colesevelam, ezetimibe, fenofibrate, gemfibrozil, alirocumab, and/or evolocumab. In certain preferred embodiments, the cholesterol-lowering drug is an agent that increases the level or activity of Aster for example by activating the Aster promoter. In some embodiments, the agent comprises an Aster polypeptide and/or an Aster polynucleotide. In various embodiments, the Aster is Aster-A, Aster-B, and/or Aster-C. In In some embodiments, the reverse cholesterol transport activator is a liver X receptor (LXR) agonist (such as LXR-623), an activator of hepatic apoA-I (such as RVX-208, apoA-I mimetic peptides or a peroxisome proliferator-activated receptor α (PPARα) agonist (e.g., LY518764)), an inhibitor of cholesteryl ester transfer protein (CETP) (such as torcetrapib, anacetrapib, or delcetrapib), or an inhibitor of endothelial lipase (EL) (such as GSK 264220A).

### BRIEF DESCRIPTION OF THE DRAWINGS

FIGS. 1A-1H show that Aster proteins contain a lipid-binding fold. FIG. 1A: Gene expression was quantified by real-time PCR from WT (white) and LXR-null (LXRα−/− and LXRβ−/−, dark) mouse peritoneal macrophage. Cells were kept in 1% lipoprotein deficient serum (LPDS, 1%), 5 μM simvastatin and 100 μM mevalonate overnight, then treated with LXR ligand GW3965 (GW, 1 μM), or LXR ligand plus RXR ligand LG 100754 (LG, 100 nM) for 16 h. Values are means±SEM. Results are representative of three independent experiments. FIG. 1B: ChIP-Seq bedgraph of LXRα and LXRβ binding patterns in mouse immortalized macrophages at the promoter region of the Gramd1b locus of chromosome 9. Input (Ctrl) served as a control for LXR enrichment. FIG. 1C: Schematic representation of Aster-A, Aster-B, and Aster-C proteins. The N-terminal GRAM, central ASTER and transmembrane (TM) domains are indicated. FIG. 1D: Purified Aster domains (Aster-A₂₆₁₋₅₇₆, Aster-B₂₂₄₋₅₆₀, and Aster-C₂₀₆₋₅₂₈) bind to 22-NBD-cholesterol but not 6-NBD-cholesterol (right) with nanomolar affinity. Values are means±SEM. FIG. 1E: Aster-B₃₃₄₋₅₆₂ (1-10 μM) was titrated with 10-3000 nM 22-NBD-cholesterol in PBS and fluorescent ligand-binding assays were performed. FIG. 1F: Binding of [³H]cholesterol to purified Aster-B₂₂₄₋₅₆₀ was assessed using a GST-agarose-based. Protein was incubated with [³H]cholesterol in 1× PBS binding buffer containing 0.003% Triton X-100 and protein-bound cholesterol separated using GST-agarose columns. Competition assays were performed using increasing concentrations of unlabeled cholesterol as indicated. Results values are means±SD. FIG. 1G: Aster-B₃₃₄₋₅₆₂ was titrated with 22-NBD-cholesterol in the presence of vehicle, estradiol, or various hydroxycholesterol (HC) sterol competitors as indicated (10 μM). Results values are means±SD. FIG. 1H: Sucrose-loaded heavy PC/Dansyl-PE liposomes 85:15 mol/mol, 2 mM lipids, 400 nm diameter) and light PC/Dansyl-PE-Cholesterol liposomes (80:15:5 mol/mol/mol, 2 mM lipids, 100 nm diameter) were incubated with no protein (buffer) or with 5 μM albumin, pre-heated Aster-B₂₂₄₋₅₆₀ (left, 95° C.×10 min), or native Aster-A₂₆₁₋₅₇₆, Aster-B₂₂₄₋₅₆₀, and Aster-C₂₀₆₋₅₂₈ (right) for 20 min at 25° C. After centrifugation, pellet fractions were collected, and the cholesterol recovered in the heavy fraction was assessed using a cholesterol oxidase (Sigma-Aldrich) assay. Pellet fractions were normalized by dansyl-PE recovery and the cholesterol/dansyl-PE ratio was compared with that of the light liposome sample (% transfer). Purified Aster domains from Aster-A, Aster-B, and Aster-C rapidly transferred cholesterol between the artificial phospholipid bilayers. Values are means±SEM.

FIGS. 2A-2G show evolutionary conservation and tissue distribution of the Aster protein family. FIG. 2A: Simplified phylogram of Aster-B one-to-one conservation across vertebrates. The scale represents 10% amino acid divergence. Phylogram was made using ClustalW2 and protein sequences were downloaded from Uniprot. FIG. 2B: Depiction of annotated intron-exon structure of Gramd1a, Gramd1b, and Gramd1c loci in Mus musculus strain C57BL/6J assembly GRCm38. Coding exons are depicted in black. 5′ and 3′ UTRs are represented with no fill. Exons which correspond to the Gram domain, Aster domain, and Transmembrane (TM) are labeled. Scale bar represents 1 kb. FIG. 2C: Aster family expression by qPCR from tissues of C57BL/6 mice (n=5). FIG. 2D: Coomassie blue-stained SDS-PAGE gel of purified FLAG-Aster domains (Aster-A₂₆₁₋₅₇₆, Aster-B₂₂₄₋₅₆₀, Aster-C₂₀₆₋₅₂₈) and control (albumin). FIG. 2E: Aster-B₃₃₄₋₅₆₂ was titrated with 22-NBD-cholesterol in the presence of cholesterol competitor as indicated. Results values are means±SD and representative of at least three independent experiments. FIG. 2F: Comparison of NBD-cholesterol binding to Aster-A, Aster-B and StarD1. FIG. 2G: Comparison of in vitro cholesterol transport by Aster proteins and StarD1.

FIGS. 3A-3E show crystal structure of the sterol-binding domain of Aster-A. FIG. 3A: The crystal structure of the ASTER domain of the mouse Aster-A. The 25-hydroxycholesterol ligand is shown as atomic spheres. The right-hand panel is rotated 90° about the indicated axis. The ligand-binding pocket is situated between a concave beta-sheet and a long carboxy-terminal helix. FIG. 3B: Details of the 25-hydroxycholesterol ligand-binding pocket. The left-hand panel shows key sidechains within the ligand pocket that mediate interaction with the ligand. In particular, Phe405, Tyr524 and Phe525 seem to determine the orientation of the ligand and are markedly different in character from the equivalent residues in the yeast Lam proteins. FIG. 3C: Cut-away view of the surface of mouse Aster A to show the ligand-binding pocket. It is notable that the ligand is completely enclosed with the exception of an opening towards the left of the pocket. The pocket is significantly larger than the ligand beyond the C3-OH group. This additional space is occupied by a glycerol molecule in all four copies of the complex in the asymmetric unit. FIG. 3D-3E: Potential mechanism for loading cholesterol into the ASTER domain. Structural rearrangements would be essential for cholesterol to gain access to the binding pocket. This is very likely to involve the loop comprising amino acids 430-439 that wraps around the ligand. Interestingly the surface of this region of the ASTER domain is relatively non-polar in character (see labeled amino acids), but with a number of prominent basic residues. It seems likely that this region of the protein will come into contact with the negatively charged/non-polar lipid bilayer into order to facilitate both loading and unloading of the cholesterol ligand.

FIGS. 4A-4B show structural comparison of sterol binding proteins. FIG. 4A: Structure of the Aster-A sterol-binding domain showing 25-hydroxycholesterol ligand and a glycerol molecule in the binding cavity. FIG. 4B: Comparison of mouse AsterA, yeast Lam4p-SD2 and StARD5. Despite the low sequence identity (18%), the Aster and Lam domains have a very similar architecture creating a largely non-polar binding cavity for the 25-hydroxycholesterol. However, the orientation of the ligands is quite distinct. The beta-sheets are most similar between AsterA and yeast Lam4p-SD2. The three helices are somewhat different. In particular, the carbxoxy-terminal helix in Aster A is one turn longer at its amino-terminus and shorter at its carboxy-terminus compared with Lam4p-SD2.

FIGS. 5A-5C show that Asters are Integral ER Proteins that Form PM Contact Sites in Response to Cholesterol. FIG. 5A: Comparative analysis of the cellular localization of full-length (1-699), B ΔGRAM (225-669), or B GRAM (1-171) alone (indicated above) Aster-B-GFP constructs with ER marker (Sec61β) in HeLa cells imaged by live-cell confocal microscopy. Scale bar 5 μm. FIG. 5B: Analysis of Aster-B-GFP (N-terminal, 1-738) localization and PM (CellMask PM stain) in A431 cells imaged by confocal microscopy. Cells were cultured in lipoprotein-deficient serum (LPDS; 5%) or loaded with cholesterol by the addition of 200 μM cholesterol:cyclodextrin complex for 1 h. Images were taken from live cells. Scale bar 5 μm. FIG. 5C: Live-cell imaging of GFP-Aster-B localization in A431 cells stably expressing BFP-KDEL (ER marker) and Cherry-CAAX (PM marker). Arrows indicate ER tubules in close proximity to the PM containing foci of Aster-B expression. Large images, scale bar=10 μm; insets, scale bar=2 μm.

FIGS. 6A-6B show that the mode of sterol binding is not conserved between Asters and yeast Lam proteins. FIG. 6A: Orientation of the 25-hydroxycholesterol ligand. A simulated annealing composite omit map unambiguously identifies the ligand orientation in mouse Aster A (left panel—contoured at 1.0 sigma). The orientation of the 25-hydroxycholesterol ligand in mouse Aster A is markedly different from that in the distantly related yeast Lam4p (right panel—pdbcode 6bym) (Jentsch et al., 2018). The ligand is rotated by approximately 120° about its long axis such that the axial methyl groups on the cholesterol are orientated very differently. FIG. 6B: Alignment of the mammalian ASTER domains with the yeast Lam2/4 proteins. Although structurally similar, there is only limited sequence homology between the sterol-binding domains of the Aster and Lam proteins (23% identity between Aster A and Lam4p-1). Residues that are identical, have high, or low similarity are shaded from dark to light gray respectively. Residues that are only shaded in the Aster-A, -B, and C—likely determine the different orientation of the sterol and are very different in character between the Aster and Lam proteins. Asterisks with medium gray shading indicate residues lining the pocket in contact with the hydroxycholesterol. Light and dark gray asterisks indicate surface non-polar and basic residues conserved in the Aster proteins that likely mediate interaction with the phospholipid membrane to facilitate sterol exchange. FIG. 6B discloses SEQ ID NOs 14-20, respectively, in order of appearance.

FIGS. 7A-7E show that Aster-B forms ER-PM Contacts in Response to Cholesterol Loading. FIG. 7A: Live-cell imaging of GFP-Aster-B localization in A431 cells stably expressing BFP-KDEL (ER marker) and Cherry-ORP5 (ER-PM contact protein) cultured in 5% LPDS (left) or following cholesterol loading for 20 min (right). Large images, scale bar=10 μm; insets, scale bar=2 μm. FIG. 7B: Live-cell imaging of GFP-Aster-B localization in A431 cells stably expressing BFP-KDEL (ER marker) and Cherry-E-Syt2 (ER-PM contact protein) cultured in 5% LPDS (left) or following cholesterol loading for 40 min (right). Large images, scale bar=10 μm; insets, scale bar=2 FIG. 7C: Quantification of Aster-B colocalization with ER-PM contact proteins. Quantification was done by selecting a square region from the bottom plane (near the PM), thresholding the punctate structures, and calculating their pixel overlap by using a colocalization tool. N=8-12 cells per construct and treatment from 2 independent experiments. FIG. 7D: Time course of Cherry and GFP fluorescence visible in the total internal reflection fluorescence (TIRF) (basal PM-associated fluorescence) field of A431 cells expressing with GFP-Aster-B and Cherry-ORP5 after the addition of cyclodextrin-cholesterol to the media. FIG. 7E: Quantification of TIRF video imaging of stable U2OS Cherry-KDEL cells treated with control or Aster-A-specific siRNA following addition of cholesterol. Video imaging was started 40 s to 90 s after addition of 1 mM cholesterol. Images were acquired every minute. Cholesterol administration resulted in the enlargement of ER-structures in close proximity to the plasma membrane and this effect is dependent on Aster-A expression.

FIGS. 8A-8E show cholesterol-dependent movement of Aster proteins to the PM. FIG. 8A: Aster-A-GFP, Aster-B-GFP and Aster-C-GFP localization in A431 cells imaged by live cell confocal microscopy in 1% LPDS (top) or following cholesterol loading for 1 h (bottom). FIG. 8B: Live-cell imaging of GFP-Aster-A localization in A431 cells stably expressing BFP-KDEL (ER marker) and Cherry Lyn (PM marker). Arrows indicate ER tubules in close proximity to the PM containing foci of Aster-A expression. FIG. 8C: Live-cell imaging of GFP-Aster-C localization in A431 cells stably expressing BFP-KDEL (ER marker) and Cherry Lyn (PM marker). Arrows indicate ER tubules in close proximity to the PM containing foci of Aster-C expression. FIG. 8D: Live-cell imaging of GFP-Aster-B localization in A431 cells stably expressing BFP-KDEL (ER marker) and Cherry-E-Syt3 (ER-PM contact protein) cultured in 5% LPDS (left) or following cholesterol loading for 40 min (right). Large images, scale bar=10 μm; insets, scale bar=2 μm. FIG. 8E: Localization of full-length Aster-B and B ΔGRAM GFP constructs expressed in A431 cells in the presence or absence of cholesterol loading imaged by live cell confocal microscopy.

FIGS. 9A-9E show that the GRAM domain mediates cholesterol-dependent Aster recruitment. FIG. 9A: Protein-lipid overlay over purified mouse Aster-B GRAM domain with various phospholipid species. PA and PS correspond to phosphatidic acid and phosphatidylserine, respectively. FIG. 9A discloses “6xHis” as SEQ ID NO: 11. FIG. 9B: Purified Aster-B GRAM domain was incubated with sucrose-loaded heavy liposomes containing Dansyl-PE and 80-85% PC or 80-85% PS+/−5% Cholesterol. Liposomes were sedimented, washed, and analyzed by immunoblotting for associated Aster-B Gram protein (anti-His antibody). FIG. 9C: Localization of full-length and B ΔGRAM Aster-B-GFP constructs with plasma membrane (CellMask stain) expressed in HeLa cells in the presence or absence of cholesterol loading imaged by live cell confocal microscopy. FIG. 9D: Comparative analysis of localization of B GRAM-GFP in CHO-K1 cells (left) and A431cells (right) culture in LPDS (top) loaded with cholesterol (bottom). The GRAM domain is recruited to the PM in response to cholesterol-loading. PM marker: PM-cherry; ER marker: mCherry-Sec61b. Scale bar: 5 μM. Results are representative of at least five independent experiments. FIG. 9E: Quantification of Aster-B PH-EGFP intensity in the TIRF plane upon cholesterol loading (left panel; n=2-3 cells, error bars+/−SD). Representative TIRF images (right panel) from GFP and Aster-B PH domain-EGFP expressing cells at the indicated time following cholesterol loading. TIRF videos recorded 5 slices (slices 3-5 were used to calculate FO) before cholesterol addition.

FIGS. 10A-10B show development of mice lacking Aster-B. FIG. 10A: RNA-seq expression of Gramd1a, Gramd1b, and Gramd1c in female adult C57BL/6J mouse adrenal gland tissue (10 weeks or age). FIG. 10B: Strategy for generating Crispr-Cas9 mediated global Gramd1b (Aster-B) knockout mice. Coding exons are depicted in black. Exons that correspond to the Gram domain, ASTER domain, and transmembrane (TM) domain are labeled. Scale bar represents 1 kb.

FIGS. 11A-11G show that Aster-B Ablation Disrupts Adrenal Cholesterol Homeostasis. FIG. 11A: Immunoblot analysis of SR-BI and Aster-B in various tissues from 7-week-old C57BL/6J mice. HMGB1 was used as a loading control. FIG. 11B: Representative immunoblot from adrenal lysates of WT and Aster-B KO mice. FIG. 11C: Gross appearance of adrenal glands from representative 6-week-old WT and Aster-B knockout mice (1 mm scale). Results are 100% penetrant and representative of at least ten independent mice of both genders. FIG. 11D: Histological sections of the adrenal cortex from wild-type and Aster-B KO mice stained with oil red O. Representative of eight images per group; 12 μm sections; scale bar: 50 μm. FIG. 11E: Representative electron micrographs of adrenal fasciculata cells from WT and Aster-B KO adrenal sections. Samples were fixed and processed as described in the Methods. Lipid droplets, nuclei and mitochondria are indicated (LD, N, MT; N=2 mice, 35-52 sections each). FIG. 11F: ESI-MS/MS analysis of the abundance of free cholesterol in adrenal glands from WT and Aster-B KO mice (n=7). Statistical analysis was performed using Student's t-test. Values are mean±SEM. FIG. 11G: ESI-MS/MS analysis of the abundance of cholesterol ester species in adrenal glands from WT and Aster-B KO mice (N=7). Statistical analysis was performed using Student's t-test. Values are means±SEM. *p<0.05; **p<0.01; ***p<0.001; ****p<0.0001.

FIGS. 12A-12F show that Aster-B facilitates HDL-cholesterol uptake. FIG. 12A: Immunoblot validation of GFP-Aster-B and SR-BI expression compared to A431 wild-type cells. FIG. 12B: Lipid composition of HDL2 purified from human plasma by ultracentrifugation. HDL2 was used for the experiment because of higher lipid content and better SR-B1 interaction compared to HDL3. FIG. 12C: A431-SR-BI/Aster-B-GFP cells were lipid depleted for 24 h (LPDS) and treated with control medium (LPDS), 150-200 μg/mL FC-HDL2, or 200 μM cholesterol-cyclodextrin for 1 h. Cells were then fixed and imaged with TIRF microscopy to visualize Aster-B in close proximity to the PM. Dashed lines indicate individual cells. Scale bar 10 μm. FIG. 12D: Cells were automatically identified using DAPI (nuclei) and CellMask (cytoplasm) images and used to quantify single cell TIRF intensities. FIG. 12E: Quantification of the mean cellular GFP-Aster-B TIRF intensity. N=254 cells for control, 307 cells for HDL2 and 187 cells for cholesterol from 4 independent experiments. FIG. 12F: Epinephrine and dopamine levels in adrenal glands.

FIGS. 13A-13H show that Aster-B-deficient mice are defective in PM to ER cholesterol transport. FIG. 13A: Immunoblot analysis of membrane (top) and nuclear (bottom) protein levels in 3T3-L1 cells treated for 48 h with control or Aster-A-specific ASO and then for the indicated times with cyclodextrin-cholesterol. Calnexin was used as a membrane loading control and Lamin A/C as a nuclear loading control. FIG. 13B: Real-time PCR analysis of gene expression in 3T3-L1 cells treated for 48 h with control or Aster-A-specific ASO and then for the indicated times with cyclodextrin-cholesterol. ***p<0.001. FIG. 13C: Real-time PCR analysis of Abca1 expression in 3T3-L1 cells treated for 48 h with control or Aster-A-specific ASO and then for the indicated times with cyclodextrin-cholesterol. **p<0.01; ***p<0.001. FIG. 13D: Time course of cholesteryl ester formation in 3T3-L1 fibroblasts treated with control or Aster-A-specific ASO for 48 h. Cells were incubated with [³H]oleate for the indicated time, CE was isolated by TLC, and the incorporation of label into CE quantified by scintillation counting and normalized to protein (mg). *p<0.05; ***p<0.001. FIG. 13E: Expression levels of the indicated genes in adrenal glands from WT and Aster-B KO mice (mean±SEM; N=6-8 per group). Statistical analysis was performed using Student's t-test. Values are means±SEM. *p<0.05; **p<0.01; ***p<0.001; ****p<0.0001. FIG. 13F: Immunoblot analysis of membrane (top) and nuclear (bottom) protein levels in adrenals from WT and Aster-B KO mice (n=5). Calnexin was used as a membrane loading control and Lamin A/C as a nuclear loading control. FIG. 13G: Serum cholesterol levels in WT and Aster-B KO mice that were fasting for five h (mean±SEM; N=6 per group). FIG. 13H: Serum corticosterone levels measured by ELISA in WT and Aster-B KO mice that were fed ad-lib or fasted overnight (˜16 hours). N=4 per group. Statistical analysis was performed using Student's t-test. Values are mean±SEM. *p<0.05; **p<0.01; ***p<0.001; ****p<0.0001.

FIGS. 14A-14B show model of Aster function in sterol transport. FIG. 14A: Model for the recruitment of Aster-B to the plasma membrane. The GRAM domain binds phosphatidylserine and is recruited to the plasma membrane in response to cholesterol loading. Aster-B forms cholesterol-dependent ER-PM contact sites and facilitates the trafficking of cholesterol to the ER. FIG. 14B: Model showing PM to ER transport of HDL-derived cholesterol in the presence or absence of Aster-B.

FIGS. 15A-15C show that Asters showed different binding affinity to different hydroxycholesterol (HC). Aster-A (FIG. 15A), B (FIG. 15B) and C (FIG. 15C) was titrated with 22-NBD-cholesterol in the presence of vehicle or various HC sterol competitors as indicated (3 μM). Hydroxycholesterols bind to all 3 Asters but Asters showed different binding affinity to different HC. The binding affinity for Each Aster is Aster A: 25-HC>24-HC>22R-HC>20α-HC; Aster B: 22R-HC>25-HC>24-HC˜=20α-HC; Aster C: 20α-HC>25-HC>22R-HC>24-HC. Results values are means±SD.

### DETAILED DESCRIPTION OF THE INVENTION

The present invention is based, at least in part, on the characterization of the three mammalian proteins (Aster-A, -B, and -C) that bind and transfer cholesterol between membranes. Cell-based studies indicated that Aster proteins bind sterols through their ASTER domain and phosphatidylserine through their N-terminal GRAM domain. Remarkably, Aster proteins are able to sense changes in PM cholesterol and drive the formation of ER-PM contact sites when PM cholesterol levels are elevated. One member of this family, Aster-B, is highly expressed in steroidogenic tissues and is required for the ability of HDL-cholesterol to move to the ER in adrenocortical cells and to be stored as cholesterol esters. These studies outline a critical function for Aster-B in nonvesicular transport of cholesterol in mammalian cells, and raises the possibility that other members of the Aster family could play important roles in facilitating ER-PM cholesterol movement in other cell types.

Given the remarkable differences in cholesterol abundance between the plasma membrane and different cellular compartments, it seems likely that cholesterol homeostasis in mammalian cells requires the coordinated action of dedicated proteins. Dozens of candidate cholesterol-transfer proteins have been shown to be capable of mediating sterol transfer in vitro; however, assigning physiologic functions to those proteins has proven challenging. Only three intracellular cholesterol-transfer proteins have been shown to have clear physiologic functions in vivo. The Niemann Pick type C proteins 1 and 2 (NPC1, NPC2) are critical for the export of LDL-derived cholesterol from the lysosome. Mutations in either NPC1 or NPC2 lead to accumulation of lysosomal lipids in vivo, explaining the phenotypes associated with Niemann Pick Type C syndrome. Steroid Acute Regulatory Protein (StARD1) is required for the trafficking of cholesterol to the mitochondrial inner membrane. Mutations in StARD1 are the major cause of lipoid congenital adrenal hyperplasia. The lysosome forms contact sites with peroxisomes in the trafficking of LDL-derived cholesterol. The ER also makes dynamic contact sites with other organelles, and these have been proposed to facilitate the transfer of small molecules including Ca⁺⁺ and lipids between membranes. ER cholesterol content might also be sensed by NRF1. However, no mammalian transporter has yet been shown to be required for trafficking of cholesterol from the PM to the ER.

Trafficking of cholesterol between the ER and PM has long been recognized to occur through rapid nonvesicular mechanisms. Studies have emphasized the ability of the ER to sense fluctuations in PM cholesterol and to link these with regulation of the sterol-sensing SREBP-2 pathway, and more recently with NRF1. Excess free cholesterol in the PM leads to increased cholesterol in the ER, resulting first in the suppression of SREBP-2 cleavage and cholesterol biosynthesis, and at higher concentrations to the generation of cholesterol esters by ACAT enzymes. Many putative sterol-trafficking proteins have been identified by in vitro experiments. However, defining the physiologic roles of these proteins has been challenging because of the absence of clear loss-of-function phenotypes. It has been suggested that assigning functions to these proteins may be complicated by redundancy, but an alternative possibility is that the various lipid-binding proteins have not yet been associated with the correct biological function. For example, the Osh family in yeast (OSBP family in mammals) was previously proposed to mediate PM to ER sterol trafficking; however, deletion of all Osh family members did not block the trafficking, suggesting that they may perform alterative functions.

Defining the physiologic roles of mammalian START family proteins, which lack one-to-one orthologs in invertebrates, have proven particularly difficult. The founding member of the family, StARD1, is the only one that has been shown to mediate cholesterol trafficking in vivo in both mice and humans. StARD1 mutations result in defective trafficking of cholesterol to the mitochondrial inner membrane and cause a massive accumulation of cholesterol esters in the adrenal—a phenotype opposite to that elicited by Aster-B deficiency. START-like domains with homology to the ASTER domain have been previously identified through bioinformatics approaches in plants, yeast, and other lower organisms. In yeast, a family of six Ltc proteins was identified that contain combinations of one or more GRAM domains, START-like domains, and transmembrane segments. These proteins have been linked to ergosterol transport at the ER-PM, ER-mitochondria, and ER-vacuole contact sites. However, one-to-one orthologs for the Aster proteins are restricted to vertebrates, where they appear to have evolved alongside SR-BI and other proteins involved in lipoprotein metabolism. In contrast to yeast, higher organisms must move lipids between tissues to maintain systemic homeostasis. It is therefore logical that mammals would have evolved specific transporters to facilitate the movement of lipoprotein-derived cholesterol into cells. Indeed, key residues lining the ASTER domain sterol-binding pocket are not conserved in yeast STAR-like domain proteins, resulting in a distinct mode of ligand binding.

One of the most remarkable features of the Aster proteins is their ability to localize to the PM based on the level of cholesterol in the membrane. Interestingly, NPC1L1, which facilitates intestinal cholesterol absorption, also relies on a cholesterol-mediated switch (Ge et al., 2008). NPC1L1, the target of the cholesterol-lowering drug ezetimibe, is internalized with cholesterol at the PM and then is trafficked to endosomes. This effect relies on a sterol-sensing domain (SSD) in NPC1L1. The mechanism of Aster recruitment by cholesterol appears to be distinct. The Aster GRAM domain, which binds phosphatidylserine rather than cholesterol, is necessary and sufficient for PM localization in response to cholesterol loading. Phosphatidylserine is enriched on the inner leaflet of PM; hence, phosphatidylserine binding by the GRAM domain could mediate PM localization of Asters.

While ER-PM junctions are stable and prominent in yeast, they appear to have more dynamic and tissue-adapted roles in mammalian systems (e.g., STIM1, E-Syts). Gramd1a (Aster A) are localized in ER-PM contact sites. Gramd2 has an N-terminal GRAM domain and is anchored in the ER like the Aster proteins, but lacks the central sterol-binding fold. Gramd2 may participate in cellular calcium homeostasis. The Aster proteins are unique among known ER-PM contact proteins in their ability to form membrane bridges in a cholesterol-dependent manner. Interestingly, substantial, but not complete, colocalization of Aster-B with ORP5, E-Syt2 and E-Syt3 in cholesterol-loaded cells was observed. ER-PM contacts containing Aster-B were frequently located in the same ER tubules in which ORP5, E-Syt2 or E-Syt3 were found, but some contacts appeared to contain only Aster-B, consistent with the idea that they are functionally distinct.

The trafficking of cholesterol from the PM to the ER and vice versa is likely to be critical for lipid homeostasis in many if not all cells. Furthermore, the cell type-selective expression of Aster proteins in tissues with active lipid metabolic programs shows that Asters play important roles in sterol transport and HDL metabolism in other tissues. For example, while Aster-B is expressed at low levels in the liver, other Asters are abundant. The high level of Aster-A expression in the brain shows that this family member is particularly important for sterol trafficking in neurons.

The present invention is based, at least in part, on the characterization of the three mammalian proteins (Aster-A, -B, and -C) that bind and transfer cholesterol between membranes. Aster proteins are anchored to the ER by a single transmembrane helix and facilitate the formation of ER-PM contact sites in response to cholesterol loading. The Aster proteins are the missing links in the trafficking of HDL-derived cholesterol to the ER. Studying the rodent adrenal gland, given the well-defined biological function of the SR-BI pathway in cholesterol ester storage and steroidogenesis, it is discovered that Aster-B was selectively enriched in steroidogenic organs and that its expression was required for the storage of HDL-derived cholesterol ester and steroidogenesis in the adrenal cortex. These findings elucidate a nonvesicular pathway for PM-ER sterol trafficking in mammalian cells, and they also suggest new mechanisms by which HDL-derived cholesterol is mobilized in a variety of physiological contexts.

In humans, SR-BI mutations have been linked to adrenal disease. Aster-B exists among uncharacterized proteins that are induced during the development of human adrenal glands. Based on the findings on Aster proteins presented herein, humans with adrenal disease, for example patients with congenital adrenal hyperplasia or hypocortisolism, likely have downregulation (e.g., mutation) in Aster proteins. Furthermore, the numerous links between defective cholesterol metabolism and human pathologies underscore the importance of understanding the mechanism of cellular sterol transport. Uniting the function of individual cholesterol trafficking proteins in vitro with their physiologic roles in vivo will advance our understanding of physiology and highlight opportunities to target lipid metabolism in the treatment and diagnosis of human disease caused by Aster protein deactivation (e.g., diseases associated with high cholesterol).

Non-limiting examples of diseases associated with reduced Aster protein activity and/or high cholesterol are coronary heart disease, stroke, peripheral vascular disease, erectile dysfunction, diabetes, high blood pressure, cellular cholesterol overload disease (e.g., Niemann-Pick type C disease), and/or a disease related to defects in brain cholesterol metabolism (e.g., Alzheimer's disease, Huntington's disease, and Parkinson's disease). Patients with reduced Aster protein levels or activity (e.g., mutations) can benefit from a cholesterol-lowering drug, reducing cholesterol uptake from diet, and/or a reverse cholesterol transport activator. Non-limiting examples of cholesterol-lowering drugs are atorvastatin, fluvastatin, lovastatin, pitavastatin, pravastatin, rosuvastatin, simvastatin, niacin, colestipol, cholestyramine, colesevelam, ezetimibe, fenofibrate, gemfibrozil, alirocumab, and evolocumab. Similarly, an agent that increases the level or activity of one or more Aster proteins (e.g., a compound that activates an Aster promoter, an Aster polypeptide, and/or a polynucleotide encoding an Aster polypeptide) can be used as a cholesterol-lowering drug.

Reverse cholesterol transport (RCT) is a pivotal pathway involved in the return of excess cholesterol from peripheral tissues to the liver for excretion in the bile and eventually the feces. RCT from macrophages in atherosclerotic plaques (macrophage RCT) is a critical mechanism of anti-atherogenecity of high-density lipoproteins (HDL). In this paradigm, cholesterol is transferred from arterial macrophages to extracellular HDL through the action of transporters such as ATP-binding cassette transporter A1 (ABCA1) and ATP-binding cassette transporter G1 (ABCG1). Non-limiting examples of the reverse cholesterol transport activators are liver X receptor (LXR) agonists (e.g., LXR-623), activators of hepatic apoA-I (e.g., RVX-208, apoA-I mimetic peptides and/or PPARα agonist like LY518764), inhibitors of cholesteryl ester transfer protein (CETP) (e.g., torcetrapib, anacetrapib, and delcetrapib), or inhibitors of endothelial lipase (EL) (e.g., GSK 264220A).

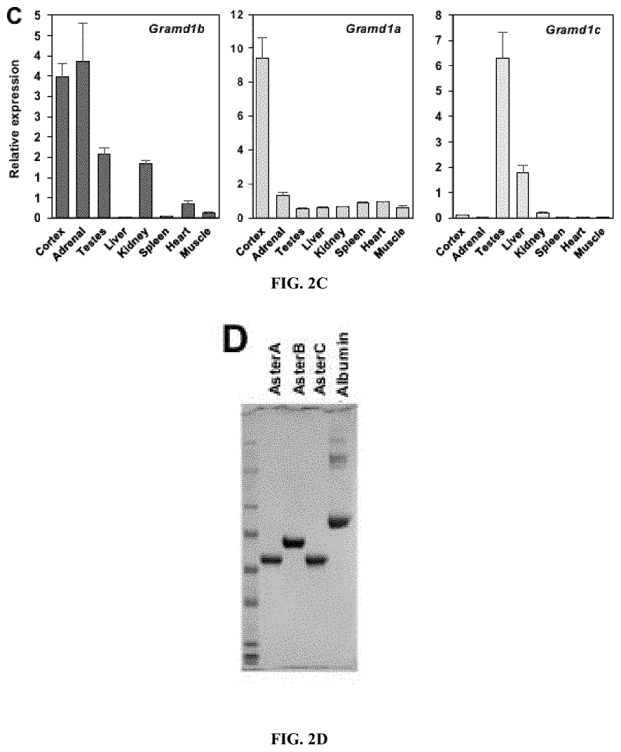

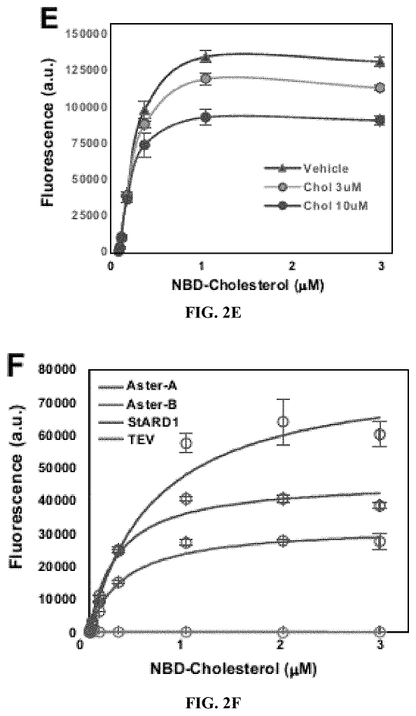

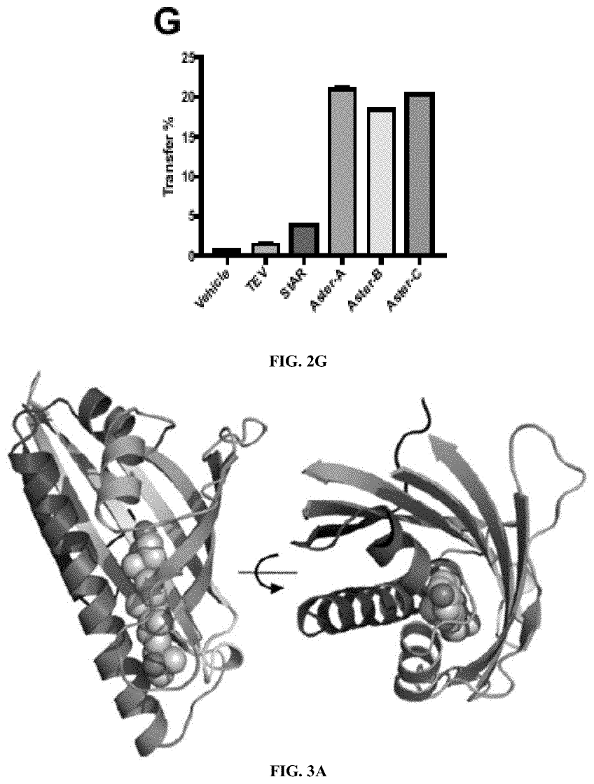

The term “Aster”, also known as Gramd1 protein, refers to previously uncharacterized proteins (Aster-A, Aster-B, and/or Aster-C) that bind and transfer cholesterol between membranes. Aster proteins are anchored to the ER by a single transmembrane helix and facilitate the formation of ER-PM contact sites in response to cholesterol loading. Gramd1b belongs to a family of highly-conserved genes, which have been designated Gramd1a, -b, and -c in databases. The predicted amino acid sequence of the human protein is 78% identical to that from Oreochromis niloticus (niletilapia). These genes have not previously been characterized; their structures are incorrectly annotated in databases; and the function of their protein products is unknown. The correct exon-intron structures of Gramd1a, -b, and -c (FIG. 2B), which are predicted to encode proteins of 723 (Aster-A), 699 (Aster-B), and 662 (aster-C) amino acids, respectively. The term “Aster” is intended to include fragments, variants (e.g., allelic variants), and derivatives thereof. Representative sequences of Aster orthologs are presented below in Table 1.

TABLE 1
SEQ ID NO: 1 Human Aster-A Amino Acid Sequence

| | |
|---|---|
| 1 | MFDTTPHSGR STPSSSPSLR KRLQLLPPSR PPPEPEPGTM VEKGSDSSSE |
| | KGGVPGTPST |
| 61 | QSLGSRNFIR NSKKMQSWYS MLSPTYKQRN EDFRKLFSKL PEAERLIVDY |
| | SCALQREILL |
| 121 | QGRLYLSENW ICFYSNIFRW ETTISIQLKE VTCLKKEKTA KLIPNAIQIC |
| | TESEKHFFTS |
| 181 | FGARDRCFLL IFRLWQNALL EKTLSPRELW HLVHQCYGSE LGLTSEDEDY |
| | VSPLQLNGLG |
| 241 | TPKEVGDVIA LSDITSSGAA DRSQEPSPVG SRRGHVTPNL SRASSDADHG |
| | AEEDKEEQVD |
| 301 | SQPDASSSQT VTPVAEPPST EPTQPDGPTT LGPLDLLPSE ELLTDTSNSS |
| | SSTGEEADLA |
| 361 | ALLPDLSGRL LINSVFHVGA ERLQQMLFSD SPFLQGFLQQ CKFTDVTLSP |
| | WSGDSKCHQR |
| 421 | RVLTYTIPIS NPLGPKSASV VETQTLFRRG PQAGGCVVDS EVLTQGIPYQ |
| | DYFYTAHRYC |
| 481 | ILGLARNKAR LRVSSEIRYR KQPWSLVKSL IEKNSWSGIE DYFHHLEREL |
| | AKAEKLSLEE |
| 541 | GGKDARGLLS GLRRRKRPLS WRAHGDGPQH PDPDPCARAG IHTSGSLSSR |
| | FSEPSVDQGP |
| 601 | GAGIPSALVL ISIVICVSLI ILIALNVLLF YRLWSLERTA HTFESWHSLA |
| | LAKGKFPQTA |
| 661 | TEWAEILALQ KQFHSVEVHK WRQILRASVE LLDEMKFSLE KLHQGITVSD |
| | PPFDTQPRPD |
| 721 | DSFS |

SEQ ID NO: 2 Human Aster-B Amino Acid Sequence

| | |
|---|---|
| 1 | MVEKGSDHSS DKSPSTPEQG VQRSCSSQSG RSGGKNSKKS QSWYNVLSPT |
| | YKQRNEDFRK |
| 61 | LFKQLPDTER LIVDYSCALQ RDILLQGRLY LSENWICFYS NIFRWETLLT |
| | VRLKDICSMT |
| 121 | KEKTARLIPN AIQVCTDSEK HFFTSFGARD RTYMMMFRLW QNALLEKPLC |
| | PKELWHFVHQ |
| 181 | CYGNELGLTS DDEDYVPPDD DFNTMGYCEE IPVEENEVND SSSKSSIETK |
| | PDASPQLPKK |
| 241 | SITNSTLTST GSSEAPVSFD GLPLEEEALE GDGSLEKELA IDNIMGEKIE |
| | MIAPVNSPSL |
| 301 | DFNDNEDIPT ELSDSSDTHD EGEVQAFYED LSGRQYVNEV FNFSVDKLYD |
| | LLFTNSPFQR |
| 361 | DFMEQRRFSD IIFHPWKKEE NGNQSRVILY TITLTNPLAP KTATVRETQT |
| | MYKASQESEC |
| 421 | YVIDAEVLTH DVPYHDYFYT INRYTLTRVA RNKSRLRVST ELRYRKQPWG |
| | LVKTFIEKNF |
| 481 | WSGLEDYFRH LESELAKTES TYLAEMHRQS PKEKASKTTT VRRRKRPHAH |
| | LRVPHLEEVM |
| 541 | SPVTTPTDED VGHRIKHVAG STQTRHIPED TPNGFHLQSV SKLLLVISCV |
| | ICFSLVLLVI |
| 601 | LNMMLFYKLW MLEYTTQTLT AWQGLRLQER LPQSQTEWAQ LLESQQKYHD |
| | TELQKWREII |
| 661 | KSSVMLLDQM KDSLINLQNG IRSRDYTSES EEKRNRYH |

SEQ ID NO: 3 Human Aster-C Amino Acid Sequence

| | |
|---|---|
| 1 | MEGAPTVRQV MNEGDSSLAT DLQEDVEENP SPTVEENNVV VKKQGPNLHN |
| | WSGDWSFWIS |
| 61 | SSTYKDRNEE YRRQFTHLPD TERLIADYAC ALQRDILLQG RLYLSENWLC |
| | FYSNIFRWET |
| 121 | TISIALKNIT FMTKEKTARL IPNAIQIVTE SEKFFFTSFG ARDRSYLSIF |
| | RLWQNVLLDK |
| 181 | SLTRQEFWQL LQQNYGTELG LNAEEMENLS LSIEDVQPRS PGRSSLDDSG |
| | ERDEKLSKSI |
| 241 | SFTSESISRV SETESFDGNS SKGGLGKEES QNEKQTKKSL LPTLEKKLTR |
| | VPSKSLDLNK |
| 301 | NEYLSLDKSS TSDSVDEENV PEKDLHGRLF INRIFHISAD RMFELLFTSS |
| | RFMQKFASSR |
| 361 | NIIDVVSTPW TAELGGDQLR TMTYTIVLNS PLTGKCTAAT EKQTLYKESR |
| | EARFYLVDSE |
| 421 | VLTHDVPYHD YFYTVNRYCI IRSSKQKCRL RVSTDLKYRK QPWGLVKSLI |
| | EKNSWSSLED |
| 481 | YFKQLESDLL IEESVLNQAI EDPGKLTGLR RRRRTFNRTA ETVPKLSSQH |
| | SSGDVGLGAK |
| 541 | GDITGKKKEM ENYNVTLIVV MSIFVLLLVL LNVTLFLKLS KIEHAAQSFY |
| | RLRLQEEKSL |
| 601 | NLASDMVSRA ETIQKNKDQA HRLKGVLRDS IVMLEQLKSS LIMLQKTFDL |
| | LNKNKTGMAV |
| 661 | ES |

SEQ ID NO: 4 Mouse Aster-A Amino Acid Sequence

| | |
|---|---|
| 1 | MFDTTPHSGR SSPSSSPSLR KRLQLLPPIR PPPASEPEPG TMVEKGSDSS |
| | SEKSGVSGTL |
| 61 | STQSLGSRNF IRNSKKMQSW YSMLCPTYKQ RNEDFRKLFS KLPEAERLIV |
| | DYSCALQREI |
| 121 | LLQGRLYLSE NWICFYSNIF RWETTISIQL KEVTCLKKEK TAKLIPNAIQ |
| | ICTESEKHFF |
| 181 | TSFGARDRCF LLIFRLWQNA LLEKTLSPRE LWHLVHQCYG SELGLTSEDE |
| | DYVCPLQLNG |
| 241 | LGSPKEVGDV IALSDISPSG AADHSQEPSP VGSRRGRVTP NLSRASSDAD |
| | HGAEEDKEEQ |
| 301 | TDGLDASSSQ TVTPVAEPLS SEPTPPDGPT SSLGPLDLLS REELLTDTSN |
| | SSSSTGEEGD |
| 361 | LAALLPDLSG RLLINSVFHM GAERLQQMLF SDSPFLQGFL QQRKFTDVTL |
| | SPWSSDSKCH |
| 421 | QRRVLTYTIP ISNQLGPKSA SVVETQTLFR RGPQAGGCVV DSEVLTQGIP |
| | YQDYFYTAHR |
| 481 | YCILGLARNK ARLRVSSEIR YRKQPWSLVK SLIEKNSWSG IEDYFHHLDR |
| | ELAKAEKLSL |
| 541 | EEGGKDTRGL LSGLRRRKRP LSWRGHRDGP QHPDPDPCTQ TSMHTSGSLS |
| | SRFSEPSVDQ |
| 601 | GPGAGIPSAL VLISIVLIVL IALNALLFYR LWSLERTAHT FESWHSLALA |
| | KGKFPQTATE |
| 661 | WAEILALQKH FHSVEVHKWR QILRASVELL DEMKFSLEKL HQGITVPDPP |
| | LDTQPQPDDS |
| 721 | FP |

SEQ ID NO: 5 Mouse Aster-B Amino Acid Sequence

| | |
|---|---|
| 1 | MESLTESGVL WSLLLELDSQ SLLWYLKRLA DAPVGAECYC WHGSEKIPAV |
| | LSPTYKQRNE |
| 61 | DFRKLFKQLP DTERLIVDYS CALQRDILLQ GRLYLSENWI CFYSNIFRWE |
| | TLLTVRLKDI |
| 121 | CSMTKEKTAR LIPNAIQVCT DSEKHFFTSF GARDRTYMNM FRLWQNALLE |
| | KPLCPKELWH |
| 181 | FVHQCYGNEL GLTSDDEDYV PPDDDFNTMG YCEEIPIEEN EVNDSSSKSS |
| | IETKPDASPQ |
| 241 | LPKKSITNST LTSTGSSEAP VSFDGLPLEE EVMEGDGSLE KELAIDNIIG |
| | EKIEIMAPVT |
| 301 | SPSLDFNDNE DIPTELSDSS DTHDEGEVQA FYEDLSGRQY VNEVFNFSVD |
| | KLYDLLFTNS |
| 361 | PFLRDFMEQR RFSDIIFHPW KKEENGNQSR VILYTITLTN PLAPKTATVR |
| | ETQTMYKASQ |
| 421 | ESECYVIDAE VLTHDVPYHD YFYTINRYTL TRVARNKSRL RVSTELRYRK |
| | QPWGFVKTFI |
| 481 | EKNFWSGLED YFRHLETELT KTESTYLAEI HRQSPKEKAS KSSAVRRRKR |
| | PHAHLRVPHL |
| 541 | EEVMSPVTTP TDEDVGHRIK HVAGSTQTRH IPEDTPDGFH LQSVSKLLLV |
| | ISCVLVLLVV |
| 601 | LNMMLFYKLW MLEYTTQTLT AWQGLRLQER LPQSQTEWAQ LLESQQKYHD |
| | TELQKWREII |
| 661 | KSSVLLLDQM KDSLINLQNG IRSRDYTAES DEKRNRYH |

SEQ ID NO: 6 Mouse Aster-C Amino Acid Sequence

| | |
|---|---|
| 1 | MEGALTARQI VNEGDSSLAT ELQEEPEESP GPVVDENIVS AKKQGQSTHN |
| | WSGDWSFWIS |
| 61 | SSTYKDRNEE YRQQFTHLPD SEKLIADYAC ALQKDILVQG RLYLSEKWLC |
| | FYSNIFRWET |
| 121 | TISIALKNIT FMTKEKTARL IPNAIQIITE GEKFFFTSFG ARDRSYLIIF |
| | RLWQNVLLDK |
| 181 | SLTRQEFWQL LQQNYGTELG LNAEEMEHLL SVEENVQPRS PGRSSVDDAG |
| | ERDEKFSKAV |
| 241 | SFTQESVSRA SETEPLDGNS PKRGLGKEDS QSERNVRKSP SLASEKRISR |
| | APSKSLDLNK |
| 301 | NEYLSLDKSS TSDSVDEENI PEKDLQGRLY INRVFHISAE RMFELLFTSS |
| | HFMQRFANSR |
| 361 | NIIDVVSTPW TVESGGNQLR TMTYTIVLSN PLTGKYTAAT EKQTLYKESR |
| | EAQFYLVDSE |
| 421 | VLTHDVPYHD YFYTLNRYCI VRSAKQRCRL RVSTDLKYRK QPWGLIKSLI |
| | EKNSWSSLES |
| 481 | YFKKLESDLL MEESVLSQSI EDAGKHSSLR RRRRTLNRTA EPVPKLSSQR |
| | SSTDLGFEAK |
| 541 | VDVTGKRKTV DSYDTALIVV MSIFLLLLVL LNVTLFLKLS KIEHATQSFY |
| | QLHLQGEKSL |
| 601 | NLVSDRFSRT ENIQKNKDQA HRLKGVLQDS IVMLEQLKSS LIMLQKTFDL |
| | LNKNKSGVAV |
| 661 | ES |

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the term “cholesterol-related disease or disorder” includes any disease, disorder, or condition which is caused by or related to dysfunction or deficiency of lipid metabolism, including, but not limited to, lipid biosynthesis, lipid transport, triglyceride levels, plasma levels, plasma cholesterol levels or misregulation or modulation of any lipid specific pathway or activity. Lipid-related diseases or disorders include obesity and obesity-related diseases and disorders such as obesity, impaired glucose tolerance (IGT), insulin resistance, atherosclerosis, atheromatous disease, heart disease, hypertension, stroke, Syndrome X, Noninsulin Dependent Diabetes Mellitus (NIDDM, or Type H diabetes) and Insulin Dependent Diabetes Mellitus (IDDM or Type I diabetes). Diabetes-related complications to be treated by the methods of the invention include microangiopathic lesions, ocular lesions, retinopathy, neuropathy, and renal lesions. Heart disease includes, but is not limited to, cardiac insufficiency, coronary insufficiency, and high blood pressure. Other obesity-related disorders to be treated by compounds of the invention include hyperlipidemia and hyperuricernia. Yet other obesity-related diseases or disorders of the invention include cachexia, wasting, anorexia, and bulimia.

As used herein, the term “cholesterol level” refers to the level of serum cholesterol in a subject or the level of cholesterol forms such as HDL cholesterol, LDL, cholesterol, and VLDL cholesterol, etc.

As used herein, the term “low density lipoprotein” or “HDL” is defined in accordance with common usage of those of skill in the art. Generally, LDL refers to the lipid-protein complex which, when isolated by ultracentrifugation, is found in the density range d=1.019 to d=1.063.

As used herein, the term “high density lipoprotein” or “HDL” is defined in accordance with common usage of those of skill in the art. Generally “HDL” refers to a lipid-protein complex which, when isolated by ultracentrifugation, is found in the density range of d=1.063 to d=1.21.

As used herein, the term “dietary constituents” includes any component of food and drink consumed by an organism, e.g., a mammal. Dietary constituents include, but are not limited to, lipids including, for example, cholesterol, e.g., LDL, VLDL, and HDL, dietary fat, fatty acids, e.g., saturated fatty acids, unsaturated fatty acids, trans fatty acids, fiber, carbohydrate, protein, amino acids, vitamins and/or minerals.

As used herein, the term “inducible” promoter is a nucleotide sequence which, when operably linked with a polynucleotide which encodes or specifies a gene product, causes the gene product to be produced in a living human cell substantially only when an inducer which corresponds to the promoter is present in the cell.

As used herein, the term “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

Modulators of Aster expression are identified in a method wherein a cell is contacted with a candidate compound and the expression of mRNA or protein, corresponding to a Aster in the cell, is determined. The level of expression of mRNA or protein in the presence of the candidate compound is compared to the level of expression of mRNA or protein in the absence of the candidate compound. The candidate compound can then be identified as a modulator of Aster expression based on this comparison. For example, when expression of Aster mRNA or protein is greater (statistically significantly greater) in the presence of the candidate compound than in its absence, the candidate compound is identified as a stimulator/activator of Aster mRNA or protein expression. Conversely, when expression of Aster mRNA or protein is less (statistically significantly less) in the presence of the candidate compound than in its absence, the candidate compound is identified as an inhibitor of Aster mRNA or protein expression. The level of Aster mRNA or protein expression in the cells can be determined by methods described herein for detecting Aster mRNA or protein.

Additionally, gene expression patterns may be utilized to assess the ability of a compound to modulate Aster e.g., by causing increased Aster expression or activity. Thus, these compounds would be useful for treating, preventing, or assessing a cholesterol-related disease or disorder. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, Gramd1 gene sequences may be used as probes and/or PCR primers for the generation and corroboration of such gene expression profiles.

### Methods of Treatment

The present invention provides for both prophylactic and therapeutic methods of treating or preventing a cholesterol-related disease or disorder in a subject, e.g., a human, at risk of (or susceptible to) a cholesterol-related disease or disorder, by administering to said subject a Aster modulator, such that the lipid-related disease or disorder is treated or prevented. In a preferred embodiment, which includes both prophylactic and therapeutic methods, the Aster modulator is administered by in a pharmaceutically acceptable formulation. Provided herein are methods of increasing the level or activity of Aster in a cell, comprising contacting the cell with an agent, wherein the agent activates the Aster promoter, comprises an Aster polypeptide, comprises an Aster polynucleotide.

The term “Aster”, also known as Gramd1 protein, refers to previously uncharacterized proteins (Aster-A, Aster-B, and/or Aster-C) that bind and transfer cholesterol between membranes. Aster proteins are anchored to the ER by a single transmembrane helix and facilitate the formation of ER-PM contact sites in response to cholesterol loading. Gramd1b belongs to a family of highly-conserved genes, which have been designated Gramd1a, -b, and -c in databases. The predicted amino acid sequence of the human protein is 78% identical to that from Oreochromis niloticus (niletilapia). These genes have not previously been characterized; their structures are incorrectly annotated in databases; and the function of their protein products is unknown. The correct exon-intron structures of Gramd1a, -b, and -c (FIG. 2B), which are predicted to encode proteins of 723 (Aster-A), 699 (Aster-B), and 662 (aster-C) amino acids, respectively. The term “Aster” is intended to include fragments, variants (e.g., allelic variants), and derivatives thereof.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the Aster molecules of the present invention or Aster modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for treating or preventing a cholesterol-related disease or disorder by administering to a subject an agent which modulates Aster expression or Aster activity. The invention also provides methods for modulating cholesterol transport, cholesterol biosynthesis, plasma triglyceride levels and plasma cholesterol levels in a subject. Subjects at risk for a cholesterol-related disease or disorder can be identified by, for example, any or a combination of the diagnostic or prognostic assays described herein. Administration of a prophylactic agent can occur prior to the manifestation of symptoms characteristic of a lipid-related disease or disorder, such that the cholesterol-related disease or disorder or symptom thereof, is prevented or, alternatively, delayed in its progression. Depending on the type of Aster aberrancy, for example, an Aster agonist or Aster antagonist agent can be used for treating the subject. The appropriate agent can be determined based on screening assays described herein.

B. Therapeutic Methods

The present invention provides methods for modulating Aster in a subject by administering an Aster modulator to either induce or inhibit Aster expression or activity. In one embodiment, Aster expression or activity is increased by administering an inhibitor or antagonist of Aster expression or activity, thereby modulating cholesterol transport, lipid biosynthesis, plasma triglyceride levels and plasma cholesterol levels in a subject and treating or preventing a cholesterol-related disease or disorder

Accordingly, another aspect of the invention pertains to methods of modulating Aster expression or activity for therapeutic purposes and for use in treatment of a cholesterol-related disease or disorder. In one embodiment, the modulatory method of the invention involves contacting a cell with an Aster polypeptide or agent that modulates one or more of the activities of Aster protein activity associated with a cholesterol-related disease or disorder (e.g., modulation of lipid biosynthesis, cholesterol transport, plasma triglyceride levels, plasma cholesterol levels). An agent that modulates Aster protein activity can be an agent as described herein, such as a nucleic acid or a protein, an siRNA targeting Aster mRNA, a naturally-occurring target molecule of an Aster protein (e.g., an Aster ligand or substrate), an Aster antibody, an Aster agonist or antagonist, a peptidomimetic of an Aster agonist or antagonist, or other small molecule. In one embodiment, the agent stimulates one or more Aster activities. Examples of such stimulatory agents include active Aster protein, a nucleic acid molecule encoding Aster, or a small molecule agonist, or mimetic, e.g., a peptidomimetic. In another embodiment, the agent inhibits one or more aster activities. Examples of such inhibitory agents include antisense Aster nucleic acid molecules, siRNA molecules, anti-Aster antibodies, small molecules, and Aster inhibitors. These modulatory methods can be performed in vitro (e.g., by culturing the cell with the agent) or, alternatively, in vivo (e.g., by administering the agent to a subject). In one embodiment, the method involves administering an agent (e.g., an agent identified by a screening assay described herein), or combination of agents that modulates (e.g., upregulates or downregulates) Aster expression or activity. In another embodiment, the method involves administering an Aster protein or nucleic acid molecule as therapy to compensate for reduced, aberrant, or unwanted Aster expression or activity.

### (i) Methods for Increasing Aster Expression, Synthesis, or Activity

As discussed above, increasing aster expression or activity may be desirable in certain situations, e.g., to treat or prevent Cholesterol-related diseases or disorders. A variety of techniques may be used to increase the expression, synthesis, or activity of Gramd1 genes and/or Aster proteins. For example, an Aster protein may be administered to a subject. Any of the techniques discussed below may be used for such administration. One of skill in the art will readily know how to determine the concentration of effective, non-toxic doses of the Aster protein, utilizing techniques such as those described below.

Additionally, RNA sequences encoding an Aster protein may be directly administered to a subject, at a concentration sufficient to produce a level of Aster protein such that Aster is modulated. Any of the techniques discussed below, which achieve intracellular administration of compounds, such as, for example, liposome administration, may be used for the administration of such RNA molecules. The RNA molecules may be produced, for example, by recombinant techniques such as those described herein. Other pharmaceutical compositions, medications, or therapeutics may be used in combination with the Aster agonists. Further, subjects may be treated by gene replacement therapy, resulting in permanent modulation of Aster. One or more copies of a Gramd1 gene, or a portion thereof, that directs the production of a normal Aster protein with Aster function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of Gramd1 gene sequences into human cells. Furthermore, expression or activity of transcriptional activators which act upon Aster may be increased to thereby increasing expression and activity of Aster. Small molecules which induce Aster expression or activity, either directly or indirectly may also be used. In one embodiment, a small molecule functions to disrupt a protein-protein interaction between Aster and a target molecule or ligand, thereby modulating, e.g., increasing or decreasing the activity of Aster.

Cells, preferably, autologous cells, containing Aster expressing gene sequences may then be introduced or reintroduced into the subject. Such cell replacement techniques may be preferred, for example, when the gene product is a secreted, extracellular gene product.

Moreover, multiple CRISPR constructs for increasing Aster expression can be found in the commercial product lists of the referenced companies, such as CRISPR products #sc-408929-ACT, sc-408929-ACT-2, sc-408929-LAC, sc-408929-LAC-2, sc-408934-ACT, sc-408934-ACT-2, sc-408934-LAC, sc-408934-LAC-2, sc-412329-ACT, sc-412329-ACT-2, sc-412329-LAC, sc-412329-LAC-2, sc-424667-ACT, sc-424667-ACT-2, sc-424667-LAC, sc-424667-LAC-2, sc-431521-ACT, sc-431521-ACT-2, sc-431521-LAC, sc-431521-LAC-2, sc-433439-ACT, sc-433439-ACT-2, sc-433439-LAC, and sc-433439-LAC-2 (Santa Cruz Biotechnology), etc.

C. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining Aster protein and/or nucleic acid expression as well as Aster activity, in the context of a biological sample (e.g., blood, serum, fluid, cells, e.g., hepatocytes, or tissue, e.g., liver tissue) to thereby determine whether an individual is afflicted with cholesterol-related disease or disorder cholesterol-related disease or disorder has a risk of developing a cholesterol-related disease or disorder. The invention also provides for prognostic (or predictive) assays for determining whether an individual is at risk of developing a lipid-related disease or disorder. For example, mutations in a Gramd1 gene can be assayed for in a biological sample. Such assays can be used for prognostic or predictive purpose to thereby phophylactically treat an individual prior to the onset of a lipid-related disease or disorder.

One particular embodiment includes a method for assessing whether a subject is afflicted with a cholesterol-related disease or disorder has a risk of developing a lipid-related disease or disorder comprising detecting the expression of the Gramd1 gene or the activity of Aster in a cell or tissue sample of a subject, wherein a decrease in the expression of the Gramd1 gene or a decrease in the activity of Aster indicates the presence of a cholesterol-related disease or disorder or the risk of developing a cholesterol-related disease or disorder in the subject. In this embodiment, subject samples tested are, for example, (e.g., blood, serum, fluid, cells, e.g., hepatocytes, or tissue, e.g., liver tissue).

Another aspect of the invention pertains to monitoring the influence of Aster modulators on the expression or activity of Aster in clinical trials.

D. Prognostic and Diagnostic Assays

To determine whether a subject is afflicted with a cholesterol-related disease or disorder or has a risk of developing a cholesterol-related disease or disorder, a biological sample may be obtained from a subject and the biological sample may be contacted with a compound or an agent capable of detecting an Aster protein or nucleic acid (e.g., mRNA or genomic DNA) that encodes an Aster protein, in the biological sample.

A preferred agent for detecting Aster mRNA or genomic DNA is a labeled nucleic acid probe capable of hybridizing to Aster mRNA or genomic DNA.

In another embodiment, the methods further involve obtaining a control biological sample from a control subject, contacting the control sample with a compound or agent capable of detecting Aster protein, mRNA, or genomic DNA, such that the presence of Aster protein, mRNA or genomic DNA is detected in the biological sample, and comparing the presence of Aster protein, mRNA or genomic DNA in the control sample with the presence of Aster protein, mRNA or genomic DNA in the test sample.

Analysis of one or more Aster polymorphic regions in a subject can be useful for predicting whether a subject has or is likely to develop a cholesterol-related disease or disorder. In preferred embodiments, the methods of the invention can be characterized as comprising detecting, in a sample of cells from the subject, the presence or absence of a specific allelic variant of one or more polymorphic regions of a Gramd1 gene. The allelic differences can be: (i) a difference in the identity of at least one nucleotide or (ii) a difference in the number of nucleotides, which difference can be a single nucleotide or several nucleotides. The invention also provides methods for detecting differences in a Gramd1 gene such as chromosomal rearrangements, e.g., chromosomal dislocation. The invention can also be used in prenatal diagnostics.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example, a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a Gramd1 gene prior to identifying the allelic variant. Amplification can be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required-amount of amplified DNA. In preferred embodiments, the primers are located between 150 and 350 base pairs apart.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), and self-sustained sequence replication (Guatelli et al., (1989) Proc. Nat. Acad. Sci. 87:1874), and nucleic acid based sequence amplification (NAB SA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a Gramd1 gene and detect allelic variants, e.g., mutations, by comparing the sequence of the sample sequence with the corresponding reference (control) sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci. 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a Grmamd1 gene in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a Gramd1 allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of Gramd1 allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163), Saiki et al (1989) Proc. Natl cad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of Gramd1. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition, it may be desirable to introduce a novel restriction site in the region of the mutation to create cleavage-based detection (Gasparini et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and can be used to detect specific allelic variants of a polymorphic region of a Gramd1 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using, hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a Gramd1 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide presents in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al, Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For determining the identity of the allelic variant of a polymorphic region located in the coding region of a Gramd1 gene, yet other methods than those described above can be used. For example, identification of an allelic variant which encodes a mutated Aster protein can be performed by using an antibody specifically recognizing the mutant protein in, e.g., immunohistochemistry or immunoprecipitation. Antibodies to wild-type Aster or mutated forms of Aster proteins can be prepared according to methods known in the art.

Alternatively, one can also measure an activity of an Aster protein, such as binding to a Aster ligand. Binding assays are known in the art and involve, e.g., obtaining cells from a subject, and performing binding experiments with a labeled lipid, to determine whether binding to the mutated form of the protein differs from binding to the wild-type of the protein.

Antibodies directed against reference or mutant Aster polypeptides or allelic variant thereof, which are discussed above, may also be used in disease diagnostics and prognostics. Such diagnostic methods, may be used to detect abnormalities in the level of Aster polypeptide expression, or abnormalities in the structure and/or tissue, cellular, or subcellular location of an Aster polypeptide. Structural differences may include, for example, differences in the size, electronegativity, or antigenicity of the mutant Aster polypeptide relative to the normal Aster polypeptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of Aster polypeptides. In situ detection may be accomplished by removing a histological specimen from a subject, and applying thereto a labeled antibody of the present invention. The antibody (or fragment) is preferably applied by overlaying the labeled antibody (or fragment) onto a biological sample. Through the use of such a procedure, it is possible to determine not only the presence of the Aster polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-Aster polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and acquorin.

If a polymorphic region is located in an exon, either in a coding or non-coding portion of the gene, the identity of the allelic variant can be determined by determining the molecular structure of the mRNA, pre-mRNA, or cDNA. The molecular structure can be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific Gramd1 allelic variant.

Sample nucleic acid to be analyzed by any of the above-described diagnostic and prognostic methods can be obtained from any cell type or tissue of a subject. For example, a subject's bodily fluid (e.g. blood) can be obtained by known techniques (e.g., venipuncture). Alternatively, nucleic acid tests can be performed on dry samples (e.g., hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

E. Recombinant Expression Vectors and Host Cells Used in the Methods of the Invention

The methods of the invention (e.g., the screening assays and therapeutic and/or preventative methods described herein) include the use of vectors, preferably expression vectors, containing a nucleic acid encoding a Aster protein (or a portion thereof). For example, in one embodiment, a vector containing a nucleic acid encoding a Aster protein, or portion thereof, is used to deliver a Aster protein, or portion thereof, to a subject, to treat or prevent a lipid-related disease or disorder in the subject. In one embodiment, the vector containing a nucleic acid encoding a Aster protein, or portion thereof, is targeted to a specific cell type, organ or tissue, e.g., a hepatocyte as described herein.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g. tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., Aster proteins, mutant forms of Aster proteins, fusion proteins, and the like).

The recombinant expression vectors to be used in the methods of the invention can be designed for expression of Aster proteins in prokaryotic or eukaryotic cells. For example, Aster proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion-protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in Aster activity assays, (e.g., direct assays or competitive assays described in detail below), or to generate antibodies specific for Aster proteins. In a preferred embodiment, a Aster fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include liver-specific promoters (e.g., the human phenylalanine hydroxylase (hPAH) gene promoter; Mancicni and Roy, (1996) Proc. Natl. Acad. Sci. USA. 93, 728-733); neuron-specific promoters (e.g., the neurofilament promoter; Byrne and Ruddle, 1989, Proc. Natl. Acad. Sci. USA 86:5473-5477), albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Baneiji et al., 1983, Cell 33:729-740; Queen and Baltimore, 1983, Cell 33:741-748), pancreas-specific promoters (Edlund et al. 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to Aster mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagenud, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a Gramd1 nucleic acid molecule of the invention is introduced, e.g., a Gramd1 nucleic acid molecule within a recombinant expression vector or a Gramd1 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a Aster protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sanbrook et al. (Molecular Cloning. A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) an Aster protein. Accordingly, the invention further provides methods for producing a Aster protein using the host cells of the invention. In one embodiment, the method comprises culturing the host cell of the invention (into which a recombinant expression vector encoding a Aster protein has been introduced) in a suitable medium such that a Aster protein is produced. In another embodiment, the method further comprises isolating a Aster protein from the medium or the host cell.

The host cells of the invention can also be used to produce nonhuman transgenic animals. For example, in one embodiment, a host cell of the invention is a fertilized oocyte or an embryonic stem cell into which sequences encoding a polypeptide corresponding to a marker of the invention have been introduced. Such host cells can then be used to create non-human transgenic animals in which exogenous sequences encoding a marker protein of the invention have been introduced into their genome or homologous recombinant animals in which endogenous gene(s) encoding a polypeptide corresponding to a marker of the invention sequences have been altered. Such animals are useful for studying the function and/or activity of Aster, for identifying and/or evaluating modulators of Aster polypeptide activity, as well as in pre-clinical testing of therapeutics or diagnostic molecules, for marker discovery or evaluation, e.g., therapeutic and diagnostic marker discovery or evaluation, or as surrogates of drug efficacy and specificity.

F. Isolated Nucleic Acid Molecules Used in the Methods of the Invention

The nucleotide sequence of the isolated human Gramd1 cDNA and the predicted amino acid sequence of the human Aster polypeptide are shown herein.

The methods of the invention include the use of isolated nucleic acid molecules that encode Aster proteins or biologically active portions thereof, as well as nucleic acid fragments sufficient for use as hybridization probes to identify Aster-encoding nucleic acid molecules (e.g., Aster mRNA) and fragments for use as PCR primers for the amplification or mutation of Aster nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

G. Isolated Aster Proteins Used in the Methods of the Invention

The methods of the invention include the use of isolated Aster proteins, and biologically active portions thereof, as well as polypeptide fragments suitable for use as immunogens to raise anti-Aster antibodies. In one embodiment, native Aster proteins can be isolated from cells or tissue sources by an appropriate purification scheme using standard protein purification techniques. In another embodiment, Aster proteins are produced by recombinant DNA techniques. Alternative to recombinant expression, a Aster protein or polypeptide can be synthesized chemically using standard peptide synthesis techniques. In a preferred embodiment, the Aster protein used in the methods of the invention has an amino acid sequence shown herein.

### EXAMPLES

The invention now being generally described, it will be more readily understood by reference to the following examples, which are included merely for purposes of illustration of certain aspects and embodiments of the present invention, and are not intended to limit the invention.

### Example 1: Identification of a Family of Mammalian Lipid-Binding Proteins

### Cell Culture

A431, CHO-K1, 3t3-L1 and HeLa cells were obtained from the American Type Culture Collection. They have been previously verified by STR testing and were confirmed to be mycoplasma-free by regular testing. CHO-K1 and HeLa cells were transfected with Fugene 6 (Promega) following manufacturer's protocol. X-tremeGENE HP DNA or Genecellin transfection reagents were used for transfecting A431 cells. A431, 3T3-L1 and HeLa stable cells were grown in monolayer at 37 C in 5% CO2. The cells were maintained in medium A (DMEM containing 100 units/ml penicillin and 100 mg/ml streptomycin sulfate) supplemented with 10% FBS. CHO-K1 cells were grown in medium B (a 1:1 mixture of F-12K medium and Dulbecco's MEM containing 100 units/ml penicillin and 100 mg/ml streptomycin sulfate) supplemented with 10% FBS. Cholesterol-depleting medium was medium A supplemented with 1-5% lipoprotein-deficient serum (LPDS). Unless otherwise specified, cells were cholesterol loaded using 200 μM cholesterol: methyl-b-cyclodextrin (randomly methylated, Sigma C4555) complexes prepared.

### Gene Expression Analysis

Total RNA was isolated using TRIzol reagent (Invitrogen) and reverse transcribed with the iScript cDNA synthesis kit (Biorad). cDNA was quantified by real-time PCR using SYBR Green Master Mix (Diagenode) on an ABI 7900 instrument. Gene expression levels were determined by using a standard curve. Each gene was normalized to the housekeeping gene 36B4 and was analyzed in duplicate. Primers used for real-time PCR are available upon request.

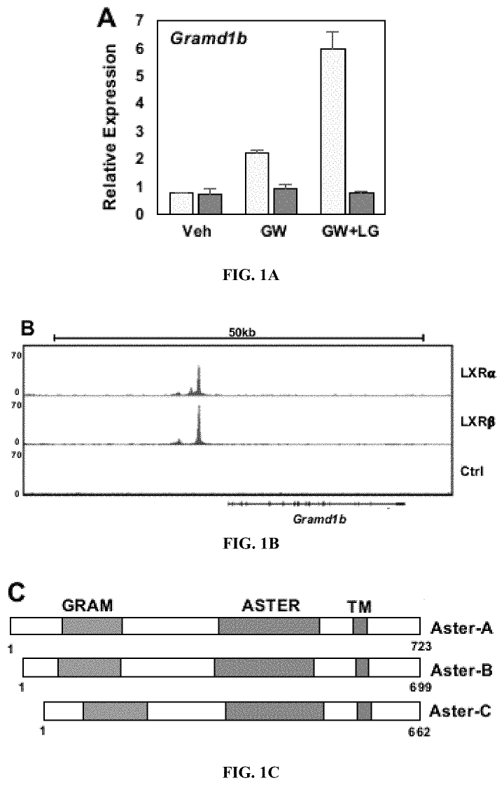

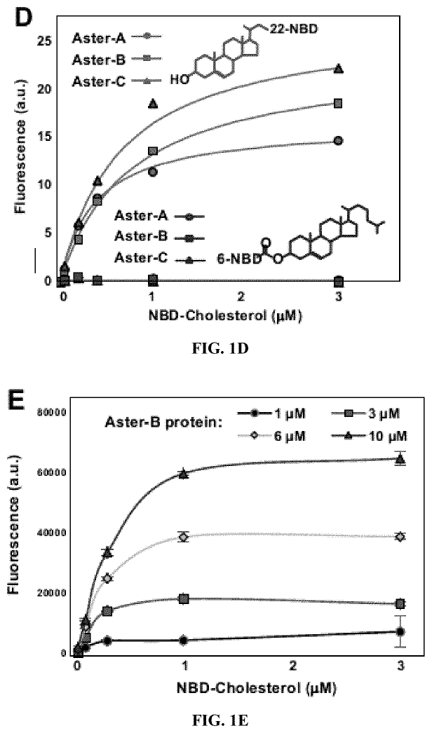

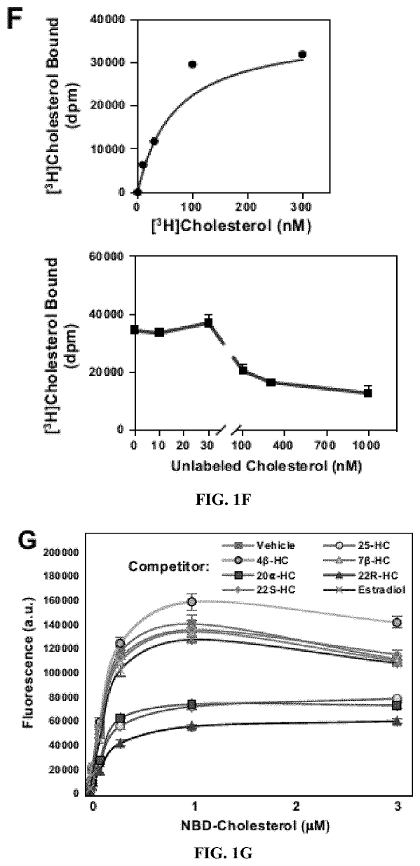

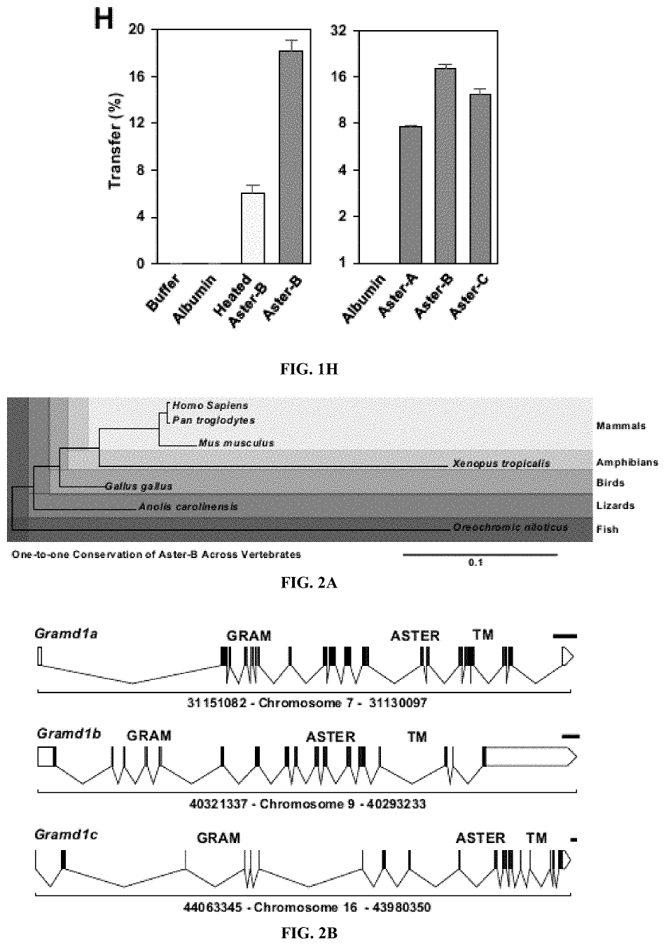

The Gramd1b gene was regulated by the sterol-responsive Liver X Receptors (LXRs) in mice, suggesting that it could play a role in sterol homeostasis. Gramd1b was induced by a synthetic LXR agonist in wild-type (WT) but not LXR-null mouse macrophages (FIG. 1A). Moreover, ChIP-seq analyses revealed binding of LXRα and LXRβ to the regulatory regions of Gramd1b, identifying it as a direct transcriptional target (FIG. 1B). Gramd1b belongs to a family of highly-conserved genes, which have been designated Gramd1a, -b, and -c in databases. One-to-one orthologs of Gramd1b are present in all vertebrate classes (FIG. 2A). The predicted amino acid sequence of the human protein is 78% identical to that from Oreochromis niloticus (nile tilapia). These genes have not previously been characterized; their structures are incorrectly annotated in databases; and the function of their protein products is unknown. The correct exon-intron structures of Gramd1a, -b, and -c are reported herein (FIG. 2B), which are predicted to encode proteins of 723, 699, and 662 amino acids, respectively. The protein products are referred to as Aster-A, -B, and -C. Real-time PCR analysis of mouse tissues revealed that the three genes are expressed in a tissue-specific manner, with Gramd1a being most abundant in the brain; Gramd1b prominently expressed in steroidogenic tissues and macrophages; and Gramd1c expressed in liver and testes (FIG. 2C).

The Aster proteins contain an N-terminal GRAM domain, which is structurally similar to a pleckstrin homology domain, and a single transmembrane domain near the C-terminus (FIG. 1C). The large central domain of the Aster proteins shows low sequence similarity to structurally characterized proteins. However, structural modeling programs such as Phyre and I-TASSER predicted that the central ASTER domain of Asters would resemble the sterol-binding domains from the mammalian StarD proteins and the Starkin domains from the yeast Lam proteins, despite minimal identity at the amino acid level. Given its similarity to the structure of the START domain, this domain was named the ASTER (Greek for “star”) domain.

### Example 2: The ASTER Domain Binds Sterols and Promotes Sterol Transfer Between Membranes

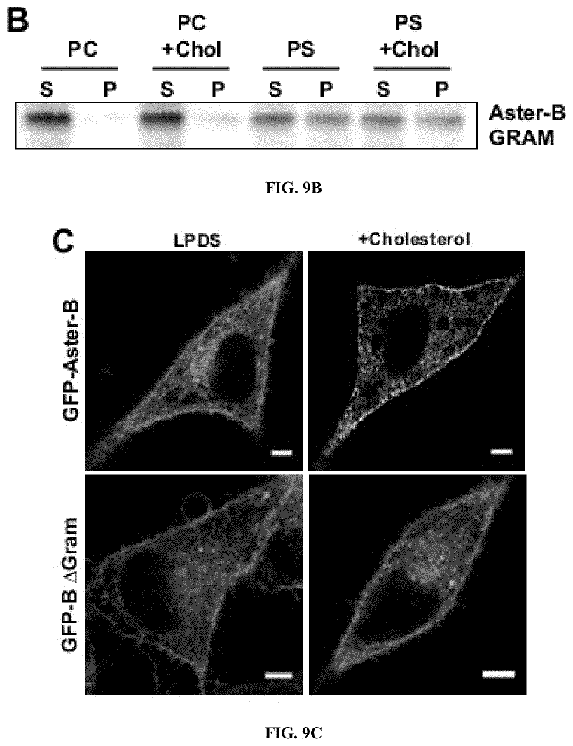

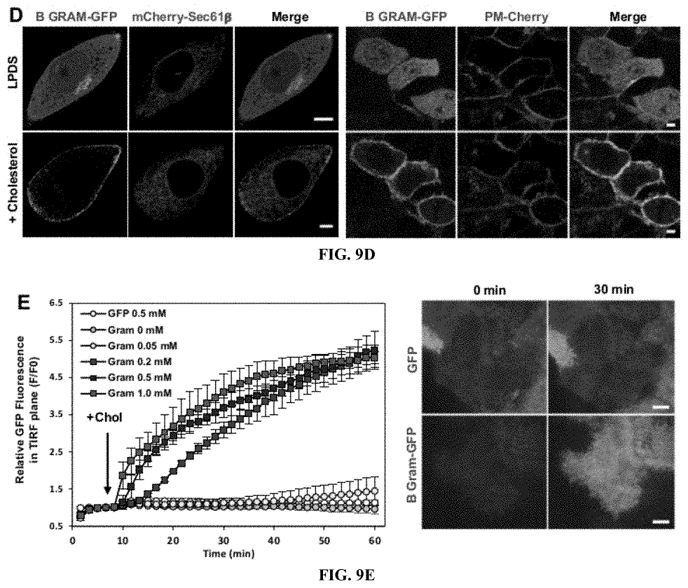

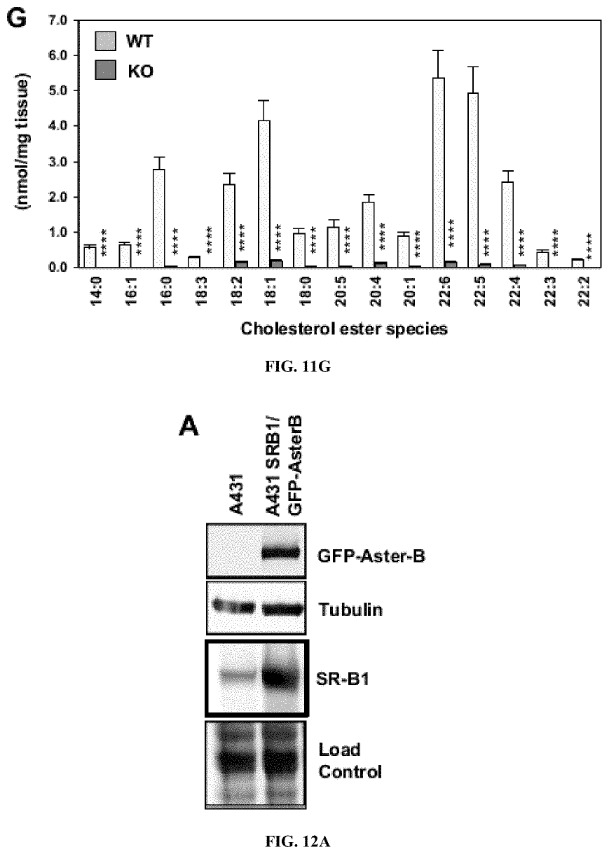

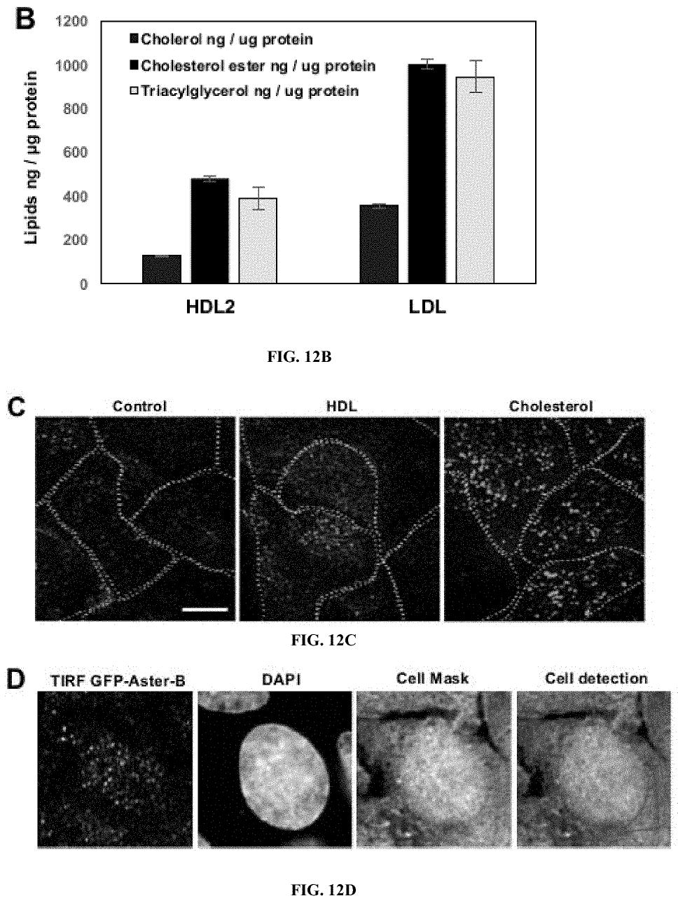

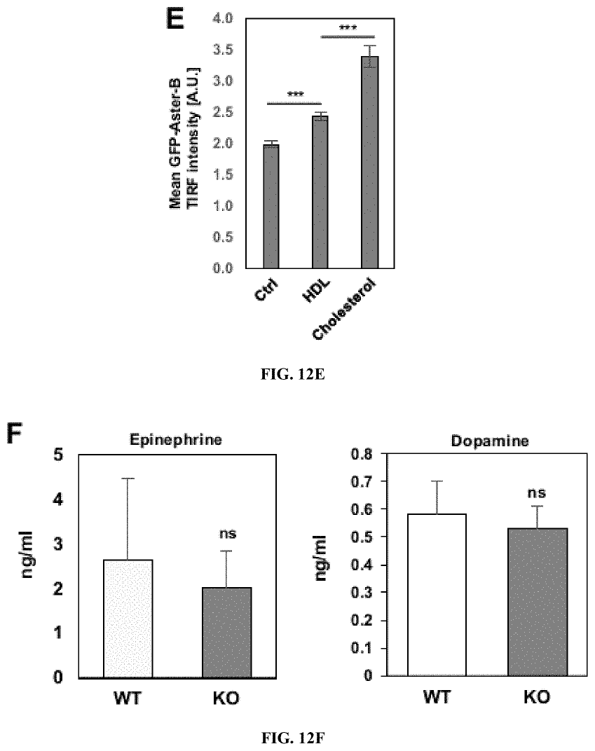

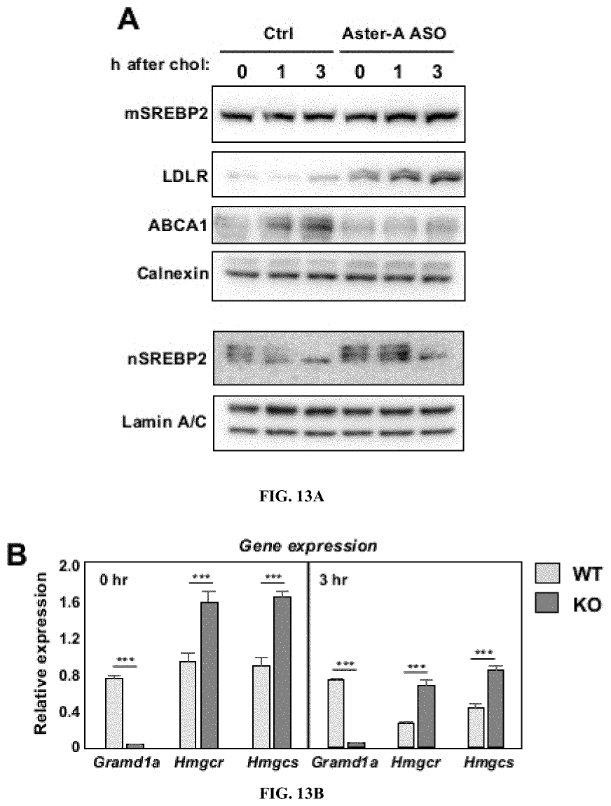

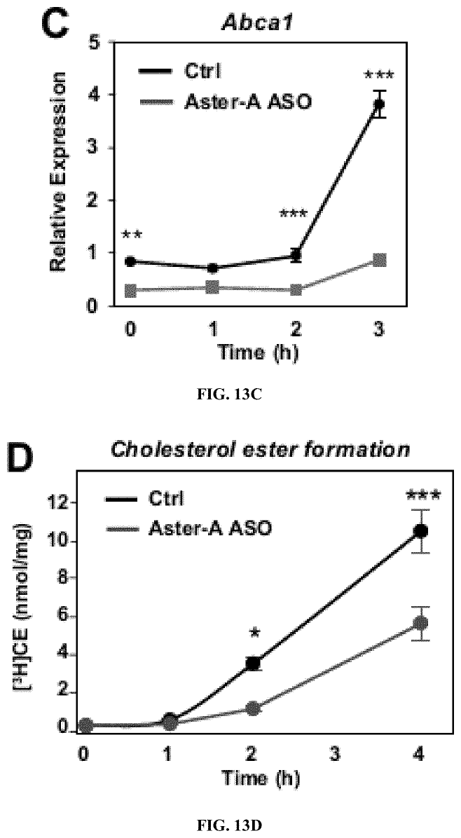

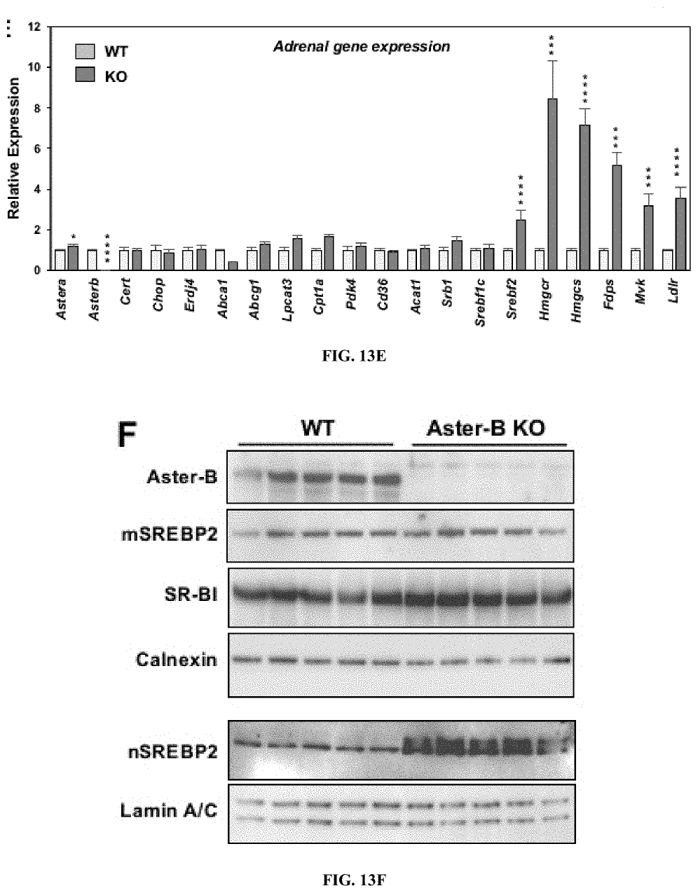

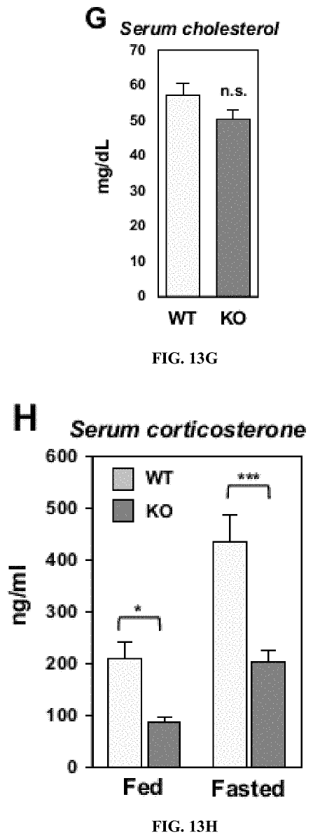

Construction of Plasmids and Stable Cell Lines Mouse Aster-A, -B, and —C and truncations were PCR amplified from Mus musculus C57BL/6J cDNA and cloned into pDonr221 by Gateway cloning (BP reaction, ThermoFisher), pEntr4-GFP-C1 or pEntr4-GFP-N2 by Gibson assembly (NEB Gibson Assembly kit). For some studies, human Aster-B (1-738) was used as indicated. For use in transient transfections of GFP-tagged Aster proteins in CHO-K1 cells, pDonr221 plasmids were LR recombined into a pDest53 destination vector containing a CMV promoter and an N-terminal eGFP. For generation of stable C-terminal GFP-tagged Aster protein A431 cells, pEntr-GFP-N2 constructs were LR recombined into a retroviral pBabe DEST vector containing an SV40 promoter. For generation of stable N-terminal GFP-tagged Aster protein A431 cells, pEntr-GFP865 C1 constructs were LR recombined into a pLenti Destination vector containing a CMV promoter. After infection, GFP positive cells were selected by flow cytometry based on expression level. PM-mCherry was generated by fusing a plasma membrane targeting sequence of GAP-43 (MLCCMRRTKQVEKNDEDQKI (SEQ ID NO: 7)) synthesized as DNA oligo (Biomers, Germany) and inserting into the N-terminus of mCherry (FIG. 9D). SR-BI cDNA (transcript variant 2, NM 001082959.1) was amplified from A431 cDNA with primers SR-BI sense-BglII (atctAGATCTaccATGGGCTGCTCCGCCAAAG (SEQ ID NO: 8)) and SR-BI anti-NotI(atttgcggccgcgtgtgtgcaggtgtgcaa (SEQ ID NO: 9)), inserted into mammalian expression vector with a EF1a promoter and puromycin selection marker (FIGS. 13A-13C and FIG. 12B). First A431 cells with a stable expression for SR-BI were selected using 1 μg/μl puromycin. These cells were then transfected with GFP-Aster-B (human) and single cell clones with stable expression of SR-BI and GFP-Aster-B were selected with 500 μg/ml G418. Aster-B PH domain-GFP and control GFP constructs were packed into retrovirus to infect A431 cells in the presence of 6 μg/ml polybrene (Millipore). Cells were selected with 1 μg/ml puromycin for 1 week before using in experiment and used as a pool without further subcloning (FIGS. 9D, and 9E).

BFP-KDEL was amplified by PCR, inserted into an AAVS1 safe harbor integration vector with puromycin selection marker. E-syt2 and E-syt3, and OSBPLS cDNA (from HeLa cDNA, NM 020896.3) amplified by PCR, and the CAAX box (KLNPPDESGPGCMSCKCVLS (SEQ ID NO: 10)) synthesized as DNA oligos (Biomers, Germany), were fused to the C-terminus of mCherry, and inserted into the safe harbor vector with blasticidin selection marker.

A431 cell lines double expressing BFP-KDEL plus the mCherry fusion proteins of interest were generated through CRISPR/Cas9 mediated AAVS1 safe harbor co-integration. Briefly, cells were co-transfected with two constructs (BFP-KDEL with puromycin selection and mCherry fusion protein with blasticidin selection) plus a third Cas9/sgRNA construct targeting the AAVS1 locus. Transfected cells were selected with 1 μg/ml puromycin and 5 μg/ml blasticidin for 7 days and used as pools for the experiments.

### Protein Expression and Purification

For crystallization studies, the mouse Aster-A protein (334-562) was cloned into pGEX2T (GE Healthcare) with a TEV protease site. Aster-A was expressed in E. Coli Rosetta (DE3) (Novagen) by growing the transformed Rosetta (DE3) at 37° C. in 2xTY until A600 nm=0.1, then inducing with 40 micromolar Isopropyl-D-1-thiogalactopyranoside (IPTG) and growth overnight at 20° C. The bacterial cells were lysed by sonication in a buffer containing 1× PBS, 1 mM Dithiothreitol (DTT) and Complete EDTA-free protease inhibitor (Roche). The soluble protein was bound to glutathione sepharose (GE healthcare), and washed with a buffer containing 1× PBS, 0.5% Triton X-100, 0.5 mM TCEP. Then the bound protein was washed with TEV cleavage buffer containing 50 mM Tris/Cl pH 7.5, 100 mM NaCl, 5% glycerol and 0.5 mM TCEP. The protein was eluted from the resin using TEV protease. After the GST purification excess 25-hydroxy cholesterol was added from a 10 mM stock solution dissolved in ethanol and the complex purified on a Superdex S-200 column in 50 mM Tris/Cl pH 7.5, 100 mM NaCl and 0.5 mM TCEP. The peak fractions were concentrated to 11.1 mg/ml and used for the crystallization experiments.

For binding and transfer studies Aster domains (Aster-A₂₆₁₋₅₇₆, Aster-B₂₂₄₋₅₆₀, Aster-C₂₀₆₋₅₂₈) were expressed by baculovirus in Sf-9 insect cells with an N-terminal FLAG tag and a C-terminal 6× His Tag (“6× His Tag” disclosed as SEQ ID NO: 11). Proteins were expressed with P3 baculovirus for 48 hours at 27° C. Cells were recovered by centrifugation, lysed by sonication. Insoluble material was pelleted at 16,000×g for 40 minutes. Soluble protein was first purified using a Ni-NTA column (Qiagen, 30210) and eluted with 250 mM imidazole PBS buffer after extensive washing. Following dialysis proteins were purified using FLAG M2 affinity gel (Sigma A2220) columns and eluted with 100 μg/mL 1× FLAG peptide after washing for ten column volumes. Proteins were then either dialyzed to remove the FLAG peptide or purified further by size exclusion chromatography. The B Gram domain was expressed in Sf9 cells with a 6xHis tag (“6xHis tag” disclosed as SEQ ID NO: 11) (Aster-B₃₀₃₋₅₃₃) and purified as described above. N-terminal GST Aster-B₃₀₃₋₅₃₃ expression constructs were transformed in Rosetta 2 (DE3) cells (Novagen). LB precultures were diluted into large-scale expression cultures and grown at 37° C. to an A₆₀₀ of 0.6-0.8, then induced with 0.5 mM IPTG at 18° C. for 16 hours with shaking. Protein was then purified in PBS+0.5 mM DTT using glutathione agarose resin (Pierce PI16100) and eluted with 10 mM GSH peptide in 50 mM Tris, 150 mM NaCl, pH 8.0. Protein was then dialyzed to remove GSH peptide. Soluble StAR was first purified using a Ni-NTA column (as above) and subsequently purified using amylose resin (NEB E8021S) and eluted with 10 mM maltose in 50 mM Tris, 150 mM NaCl, pH 8.0. Protein was then dialyzed to remove maltose.

### NBD-Cholesterol Binding Experiments

Fluorescent sterol binding assays were carried out as previously described (Petrescu et al., 2001; Wei et al., 2016) in 384-well black flat-bottom plates and equilibrated at room temperature for 1 hour. Measurements were made using a CLARIOstar (BMG LABTECH) microplate reader. The NBD fluorophore was excited with 1(ex)=470 nm and 1(em)=525 and plotted using Prism software. Dissociation constants (K_{D}) were determined by nonlinear regression analysis of dose-response curves.

### GST Agarose Assay for [³H] Cholesterol Binding

Reactions were carried out in binding buffer (0.003% Triton X-100 in 1× PBS) containing 150 nM of Aster-B ASTER protein and [³H]cholesterol. After incubation for 30 min at room temperature, the mixture was incubated with pre-equilibrated of glutathione agarose resin (Pierce PI16100) at 4° C. for 2 h, then loaded onto a column and washed. The protein-bound [³H]cholesterol was eluted with 10 mM GSH peptide and quantified by scintillation counting. For competition experiments with unlabeled sterols, the assays were carried out in the presence of ethanol containing the indicated unlabeled sterol (0-10

### Liposome Preparation

Liposomes were generated by drying lipids in glass tube under liquid nitrogen. Lipid films were then resuspended in 50 mM hepes, 120 mM potassium acetate buffer ±0.75M sucrose where indicated. Lipid suspensions were then vortexed and incubated for 30 minutes at 37° C. (2 mM total lipid concentration). Suspensions were then snap frozen in liquid nitrogen and thawed rapidly at 37° C. five times. Light liposomes were prepared by extruding through 100 nm polycarbonate filters. Heavy liposomes containing sucrose were prepared by extruding through a 400 nm polycarbonate filter, then washing several times in 50 mM hepes, 120 mM potassium acetate buffer with no sucrose. Liposome sizes were confirmed using an N4 Dynamic Light Scattering instrument.

Molecular modeling of the ASTER domain indicated the presence of a hydrophobic pocket capable of accommodating a lipophilic molecule. To test their ability of the ASTER domain to bind lipids, the ASTER domains from Aster-A, -B, and -C were expressed and purified (FIG. 2D). Using a fluorescent NBD-cholesterol binding assay, it was discovered that all three Aster domains avidly bound sterols. As shown in FIG. 1D, increasing the concentration of NBD-cholesterol (over the range 10-3,000 nM) (while maintaining the concentration of the ASTER-domain constant) increased the fluorescence emission of NBD-cholesterol. Fitting the data to a single exponential yielded an average Kd of <100 nM. By contrast, 6-NBD-cholesterol, which would place the NBD inside of the binding pocket, did not bind to the ASTER domains, indicating that only certain sterol species can be accommodated (FIG. 1D). Using the Aster-B ASTER domain confirmed increased NBD fluorescence in response to increasing protein concentration (FIG. 1E). This also confirmed direct binding of [³H]cholesterol to the ASTER domain of Aster-B (FIG. 1F).

Competition studies further showed that binding of NBD-cholesterol to the ASTER domain from Aster-B was inhibited by 22-R, 25-, and 20α-hydroxycholesterol in addition to cholesterol itself (FIG. 1G and FIG. 2E). However, alternative sterols such as estradiol and 4β-, 22S-, and 7β-hydroxycholesterols were comparatively poor competitors. The inability of these oxysterols to compete for NBD-cholesterol binding suggests that the different ASTER domain binding affinities are not due to differences in the solubility of the different sterols. Finally, the affinity of the Asters for sterols was comparable to that of the sterol binding domain from the canonical START domain protein StARD1 (FIG. 2F).

To test the ability of the ASTER domain to transfer cholesterol between membranes, an in vitro assay was optimized with heavy and light liposomes. Purified recombinant ASTER domain was incubated with “heavy” PC/dansyl-PE liposomes and “light” PC/cholesterol/dansyl-PE liposomes under agitation for 15 min. The liposomes were separated by centrifugation, and cholesterol levels were determined. Dansyl-PE intensities of heavy liposomes were used to normalize the cholesterol values. The ASTER domain from Aster-A, -B, and —C, but not BSA, efficiently facilitated cholesterol transfer to the heavy liposomes (FIG. 1H). Preheating the ASTER domain to 95° C. for 10 min substantially reduced its activity. Interestingly, the ASTER domains were more efficient transporters of cholesterol in this assay than the START domain of the canonical StARD1 protein (FIG. 2G). These results conclude that the ASTER domain efficiently binds and transfers cholesterol between membranes in vitro.

### Example 3: Crystal Structure of the Aster Sterol-Binding Domain

### Crystallization and X-Ray Structure Determination

Crystals of the Aster-A (334-562):25-hydroxycholesterol complex were obtained using sitting drop vapor diffusion at room temperature. Crystals were grown using 0.2 M NaCl, 0.1 M sodium cacodylate pH 6.0 and 8% PEG 8000 (condition E3 Proplex, Molecular Dimensions). Data were collected to 2.9 Å on the 103 beamline at Diamond Light Source, UK. Data were processed using XDS (within Xia2) and Pointless/Aimless (within CCP4). The structure was solved using molecular replacement using Phaser (within CCP4) and the first StARkin domain of S. cerevisiae Lam4 (pdb code 5YQJ) as a model. Model fitting and refinement were performed using Coot, Refmac (within CCP4), PDB-REDO and Phenix.

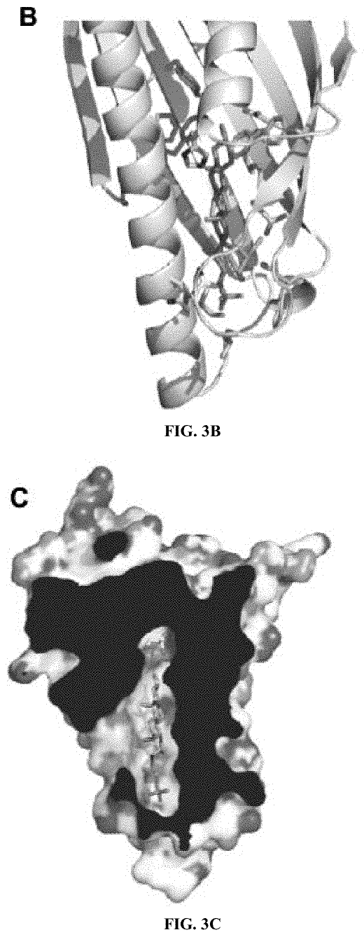

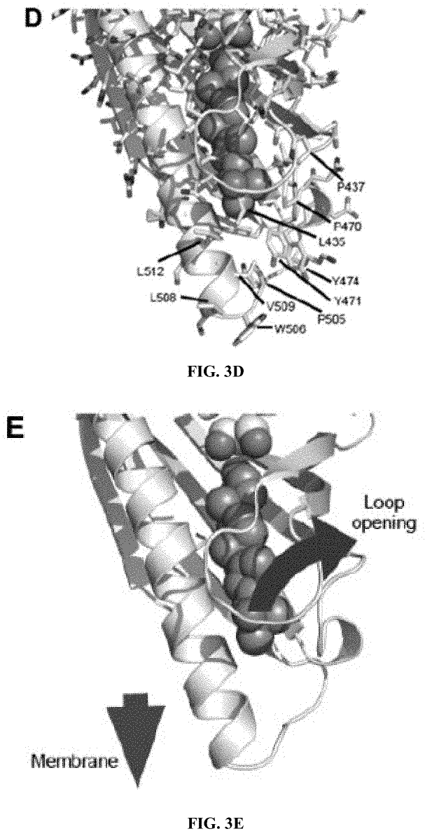

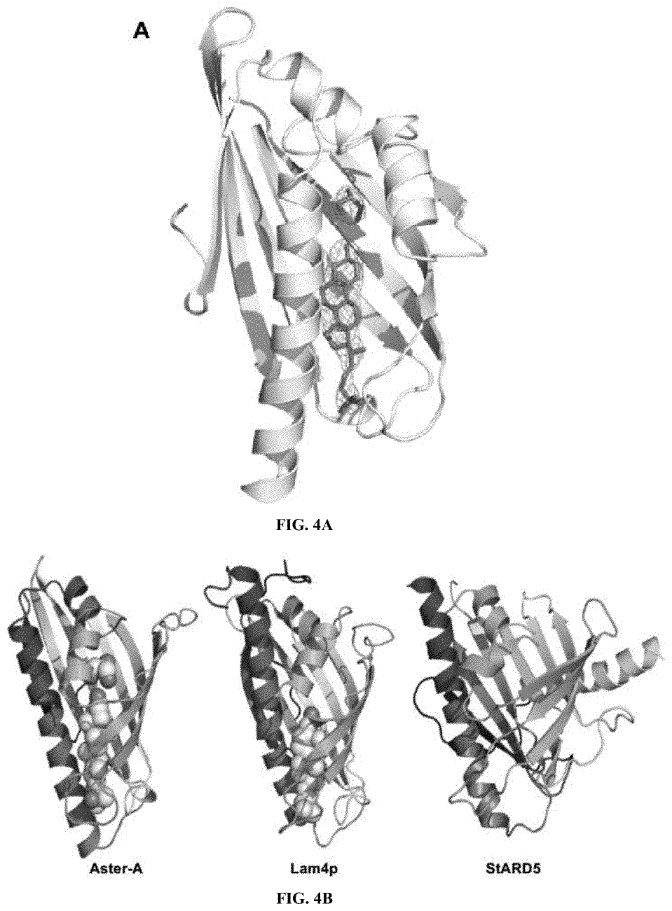

To determine the structure and to characterize the mode of sterol binding, the ASTER domain was crystalized from Aster A (aa: 334-562) with 25-hydroxycholesterol. The domain was expressed as a GST fusion protein in E. coli, purified and crystalized in the presence of 25-hydroxycholesterol. Despite the predicted similarity to the StARD and Lam proteins, solving the structure by molecular replacement proved to be challenging. A solution was found using a truncated model based on the structure of the first start domain from the yeast protein Lam4 (5YQJ) with 23% sequence identity. Overall the structure of the ASTER domain consists of a highly curved 7-stranded beta-sheet forming a groove to accommodate the hydroxycholesterol ligand. The cavity is closed by a long carboxy-terminal helix and two shorter helices following the amino-terminal beta-strand (FIG. 3A). The electron density for the 25-hydroxycholesterol unambiguously defined the position and orientation of the sterol, which was identical in all four molecules within the asymmetric unit (FIG. 3B and FIG. 4A). Interestingly, there was additional volume within the cholesterol-binding cavity adjacent to the C3-OH group of the cholesterol. Within this volume we observed electron density for a glycerol molecule adjacent to the hydroxyl group on the cholesterol (FIG. 3C and FIG. 4A). Glycerol was present during purification of ASTER domain and also used as a cryo-protectant. Interestingly the glycerol is ideally sized to fill the remaining volume of the pocket that is not occupied by the hydroxycholesterol.

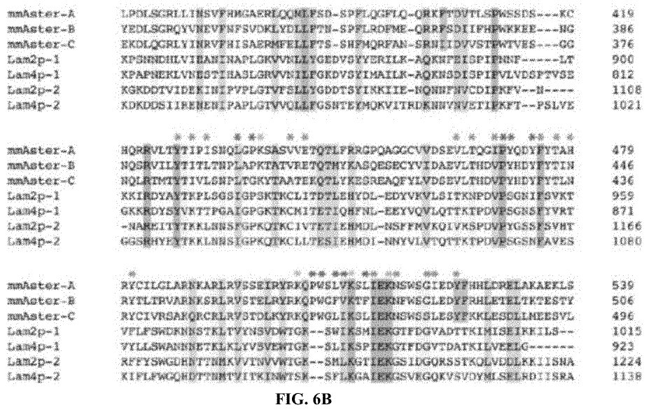

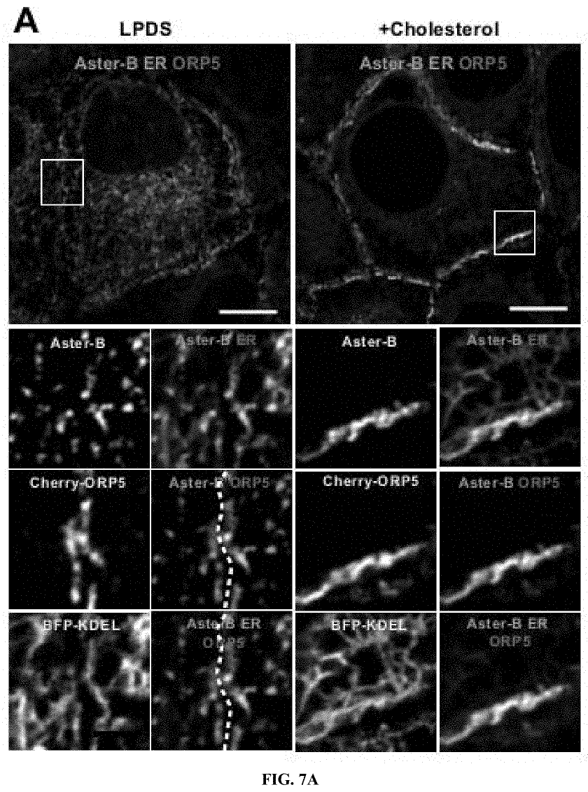

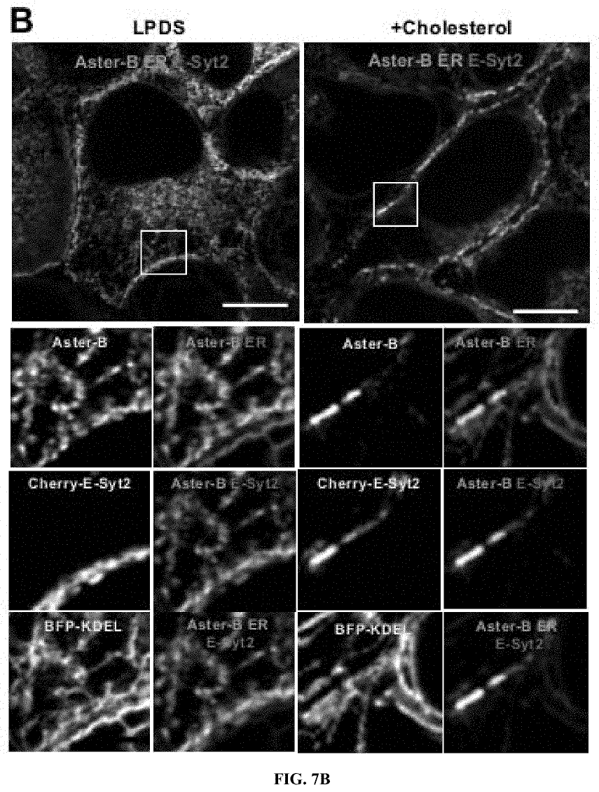

Despite the relatively low sequence identity, the three-dimensional structure of Aster A broadly resembles the START domain fold, and is even more similar to the START-like domains in the Lam2 and Lam4 proteins (C-alpha RMSD c.2 Å; FIG. 4B). However, sequence differences within the cholesterol binding pocket result in a different binding mode for the ligand, such that in Aster-A the sterol is rotated by approximately 120° about the long axis of ligand compared with the ligands in the START domains. This appears to be a concerted effect of multiple amino acid differences, but in particular F405, Y524 and F525 in mouse Aster-A seem to influence the ligand orientation (FIG. 3B, FIG. 6A, and FIG. 6B). Interestingly these residues are conserved in all 3 mammalian Aster proteins but not the yeast Lam proteins (FIG. 6B).

The sterol-binding pocket within the ASTER domain is largely enclosed with the exception of a relatively small opening adjacent to the loop between beta-strands 3 & 4. In order for the sterol to gain access to the pocket it is very likely that this loop will open (FIG. 3D and FIG. 3E). In all four complexes within the asymmetric unit, this loop has relatively high B-factors or could not be modeled, consistent with conformational flexibility. Interestingly, there is an abundance of surface-exposed non-polar residues located at the “tip” of the Aster domain around the presumed opening of the sterol-binding cavity (FIG. 3C and FIG. 3D). Alongside these non-polar residues, are a number of conserved basic residues. These give the tip of the ASTER domain an overall positive charge but with the opportunity to make non-polar interactions. This conserved surface chemistry of Aster proteins may assist exchange of sterol with negatively-charged/non-polar phospholipid membranes through interaction with and/or partial insertion of the domain into the membrane.

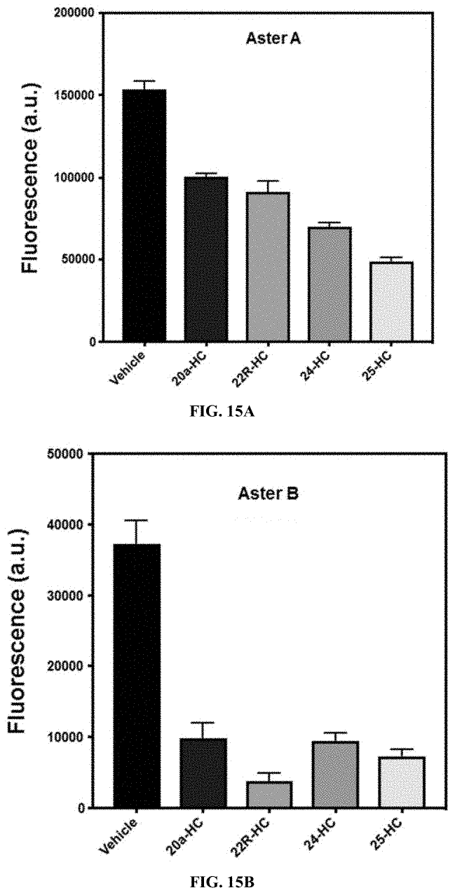

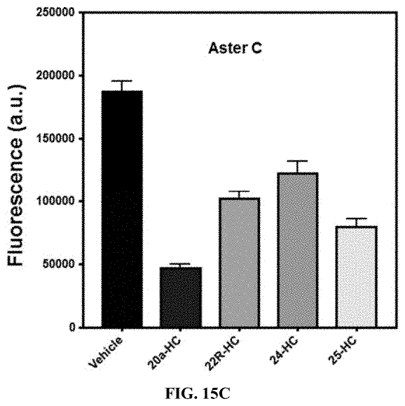

The three different Aster proteins bind different oxysterols. Therefore they could be distinguished pharmacologically; i.e. one could identify specific compounds that selectively target each Aster for tissue-specific disease intervention. FIGS. 15A-15C show that Asters showed different binding affinity to different hydroxycholesterol (HC). Aster-A (FIG. 15A), B (FIG. 15B) and C (FIG. 15C) was titrated with 22-NBD-cholesterol in the presence of vehicle or various HC sterol competitors as indicated (3 μM). Hydroxycholesterols bind to all 3 Asters but Asters showed different binding affinity to different HC. The binding affinity for Each Aster is Aster A: 25-HC>24-HC>22R-HC>20α-HC; Aster B: 22R-HC>25-HC>24-HC˜=20α-HC; Aster C: 20α-HC>25-HC>22R-HC>24-HC. Results values are means±SD.

### Example 4: Asters are Integral ER Proteins Recruited to the Plasma Membrane by Cholesterol

### Live Cell Imaging

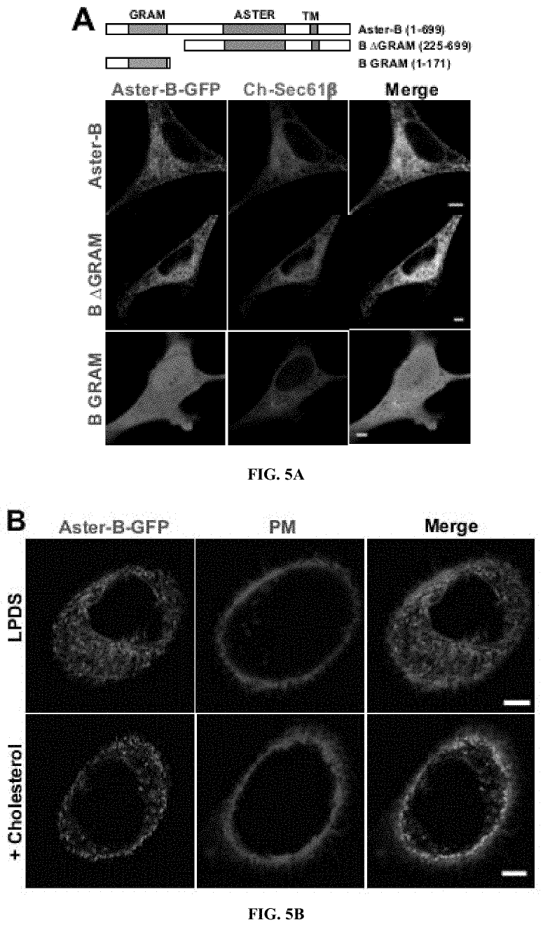

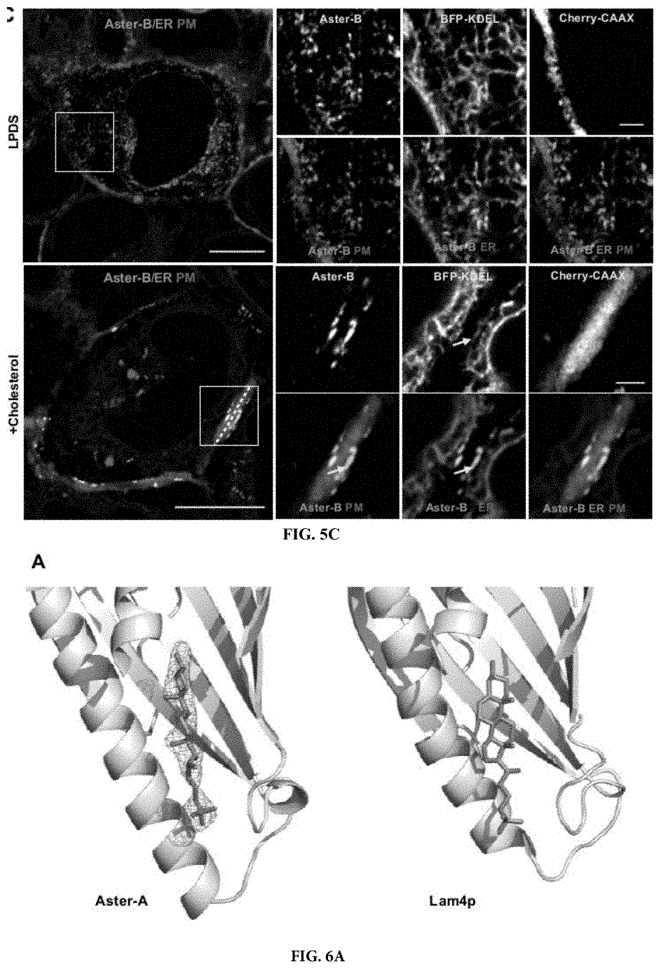

For time-lapse fluorescence imaging, cells were plated in poly-d-lysine coated 35 mm glass bottom dishes (Mat-tek) and, when indicated transfected 48 hr prior to imaging. Images were acquired using an Inverted Leica TCS-SP8-SMD Confocal Microscope, equipped with CO₂/temperature controlled Tokai Hit system for imaging of live cells at 37° C. with 5% CO₂. Images were deconvolved using Huygens Professional software. Brightness and contrast were adjusted with ImageJ software. For some experiments cells were sorted for expression of GFP. TIRF imaging was performed in glass bottom μ-slide 4 well plates (Ibidi) with a Nikon Eclipse Ti-E N-STORM microscope, equipped with Andor iXon+897 back-illuminated EMCCD camera and x 100 Apo TIRF oil objective NA 1.49, a 65 mW Argon line combined with Quad filter was used for visualization of TIRF and epifluorescence (FIG. 5E). Live cell TIRF imaging was performed similarly at 37° C., 5% CO₂ with EMBL GP168 incubator controller (FIG. 6E).

For automated quantification of TIRF images, cells were fixed with 4% PFA for 15 min, permeabilized with PBS/0.1% Triton for 5 min and stained with DAPI 5 μg/ml and 0.2 μg/ml CellMask Deep Red (Life Technologies) in PBS for 15 min. TIRF images were acquired for GFP-AsterB together with epifluorescent images for DAPI and CellMask Deep Red. These images were automatically quantified using CellProfiler and the resulting data analyzed with Python/Pandas. For Airyscan superresolution microscopy, cells in 8 well Lab-Tek™ II Chambered coverglass (Thermofisher) were imaged with a Zeiss LSM 880 confocal microscope equipped with an Airyscan detector using a 63× Plan-apochromat oil objective, NA1.4. Live cell imaging was performed at 37° C., 5% CO₂ with incubator insert PM S1 and definite focus hardware autofocus system. Images were Airyscan processed automatically using the Zeiss Zen2 software package (FIG. 5D).

For dual color live cell TIRF imaging, A431 cells stably expressing GFP-Aster-B and Cherry-ORP5 were seeded in 4-well LabTek II live cell chamber slides. After two days cells were incubated with DMEM containing 5% LPDS for 8 h. Live cell TIRF imaging was performed in FluoroBrite DMEM containing 5% LPDS: TIRF video microscopy with a frame rate of one image per minute was initiated 50 s after addition of 1 mM cholesterol/cyclodextrin. Images were acquired with a GE Deltavision OMX SR instrument equipped with a 60× Apo-N oil immersion objective. Images were deconvolved using the softWoRx 7.00 software (GE Healthcare).

For imaging Aster-B with PM and other contact site marker proteins, A431 cells stably expressing BFP-KDEL and mCherry fusion proteins of ER/PM contact site markers were seeded in 8-well LabTeK II live cell chamber slides. After 24 h cells were washed with PBS and transiently transfected with GFP-Aster-B expression constructs using X-tremeGENE HP DNA or Genecellin transfection reagents in DMEM containing 5% lipoprotein deficient serum (LPDS). After 24 h cells were switched to FluoroBrite DMEM containing 5% LPDS, with or without 100-200 μM cholesterol/cyclodextrin. Cells were imaged 15 to 65 min after cholesterol administration using a Zeiss LSM 880 Airyscan microscope equipped with a 63× Plan-Apochromat oil immersion objective. Images were Airyscan processed and brightness and contrast adjusted with ImageJ. Images from the bottom section of a cell were used for the quantification of the overlap of GFP-Aster-B with contact-site marker proteins. The images were thresholded to select GFP-Aster-B and contact site marker structures and the pixel overlap of the segmented structures was calculated with the JaCOP plugin for ImageJ. For each condition, 9-13 cells from 2 independent experiments were quantified.

For Aster-A siRNA studies, U2OS cells were transfected with mCherry-KDEL and single cell clones were selected with 500 μg/ml G418. U2OS Cherry-KDEL cells were seeded into 8-well LabTeK II live cell chamber slides and reverse transfected with 50 nM control siRNA or Aster-A siRNA (target sequence: ‘5-CACGATCTCCATCCAGCTGAA-3’ (SEQ ID NO: 12)) using HiPerfect. After 48 h cells were washed with PBS and incubated with DMEM containing 5% LPDS for 24 h. For live cell TIRF imaging, medium was changed to FluoroBrite DMEM containing 5% LPDS. TIRF video microscopy with a frame rate of one image per minute was started at 40-90 s after addition of 1 mM cholesterol/cyclodextrin. Images were acquired with a GE Deltavision OMX SR instrument equipped with a 60× Apo-N oil immersion objective. Images were deconvolved using the softWoRx 7.00 software (GE Healthcare). ER structures were segmented and the average ER structure size per cell was quantified with ImageJ.

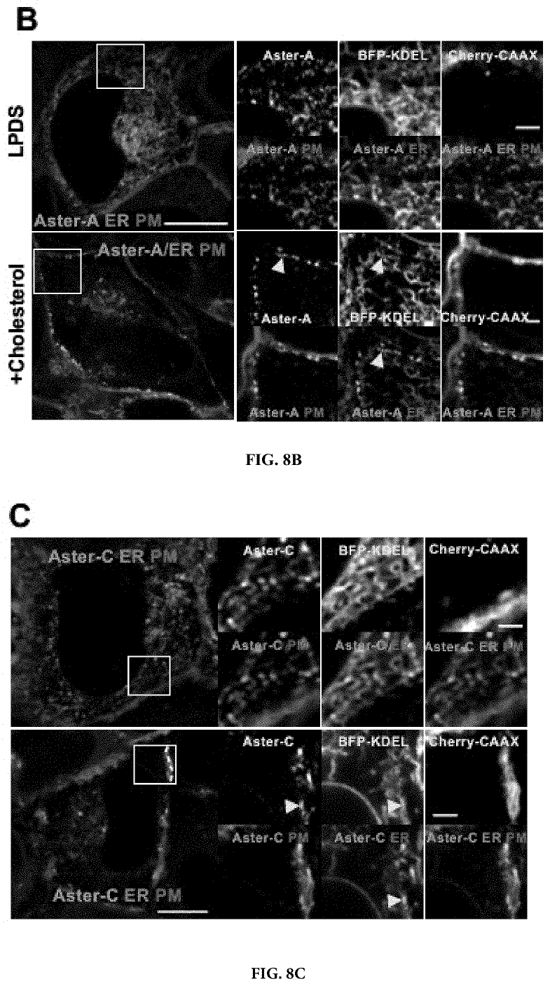

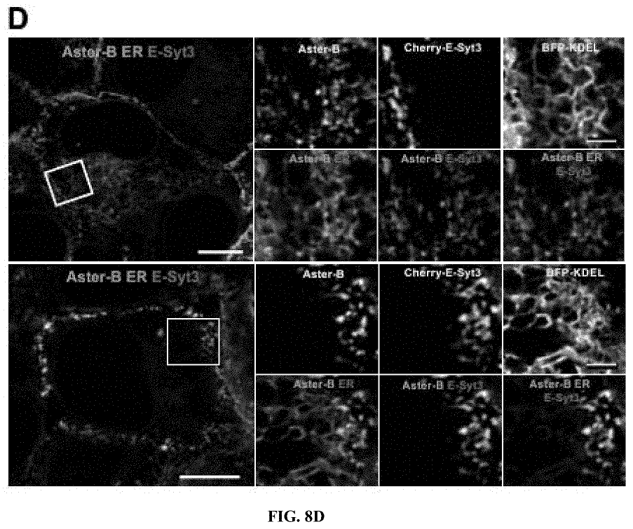

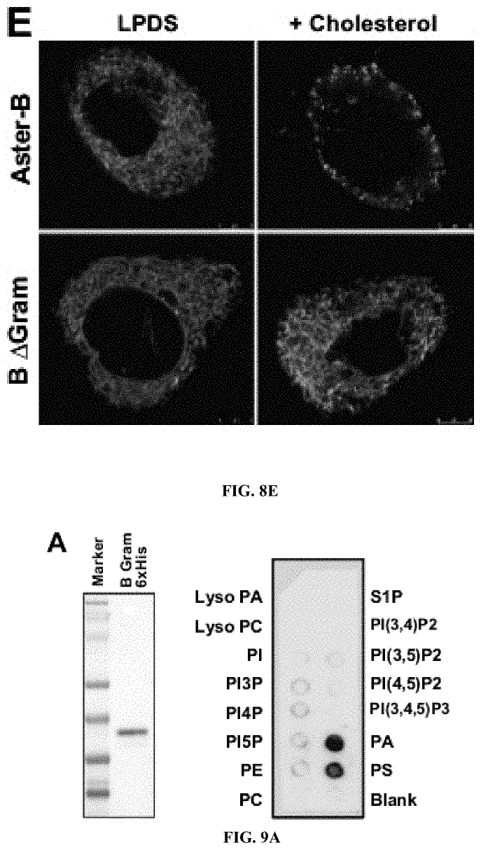

The structure of the Aster proteins suggested that they may promote the transfer of cholesterol between biological membranes. A critical question, therefore, was where these proteins are located with cells. To determine the location of Aster-A, -B, and -C, we expressed N-terminally tagged fusion proteins in A431 and HeLa cells, as detailed above. When cultured in standard lipid-poor conditions (1% lipoprotein-deficient serum, LPDS), all three Aster proteins displayed a reticular pattern that largely overlapped with the ER marker Sec61β (FIG. 5A-FIG. 5C and FIG. 8A). An Aster-B fusion protein lacking the GRAM domain was localized to the ER, but one containing the isolated GRAM domain was mainly located within the cytoplasm (FIG. 5A). This shows that the Aster proteins tethered to the ER by a single pass transmembrane helix.

The ER-localized Aster proteins might facilitate lipid transfer by making transient contacts with another cellular membrane, perhaps in response to changes in the abundance of one or more lipids. Prior work has shown that cholesterol loading by methyl-β-cyclodextrin results in rapid delivery of plasma membrane cholesterol to the ER. Remarkably, cholesterol loading also resulted in the relocalization of all 3 Aster proteins to the periphery of the cell where they appeared to be in close proximity to the PM (FIG. 5B and FIG. 8A). To explore the association of ER-anchored Aster proteins with the PM in more detail, an A431 cell line expressing both BFP-KDEL and Cherry-CAAX was generated. The location of GFP-Aster-A, -B and -C in these cells was visualized using live cell Airyscan imaging. When cells were cultured in low-cholesterol media (LPDS), all 3 Aster proteins showed a punctate pattern of distribution in ER structures throughout the cell (FIG. 5C, FIG. 8B and FIG. 8C). However, when cholesterol-cyclodextrin was added to the media, the Aster proteins were found almost exclusively in ER tubules that were in close proximity to the PM (arrows, FIG. 5E, FIG. 8B and FIG. 8C).

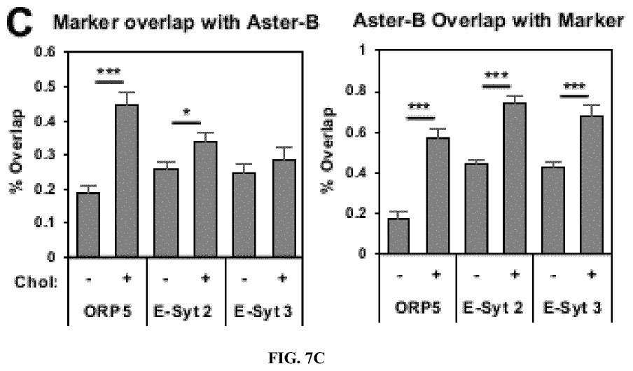

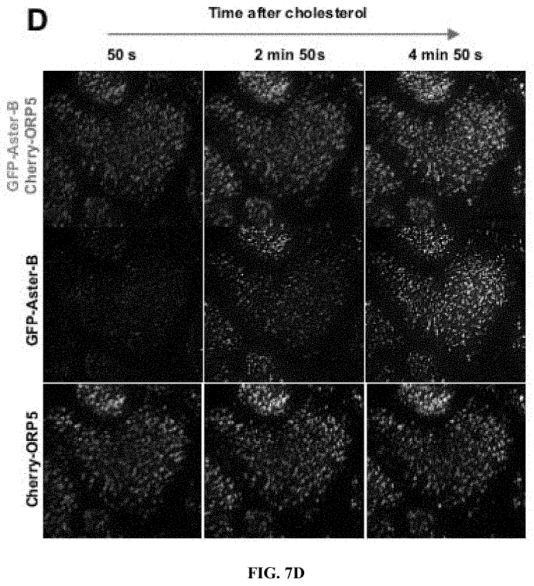

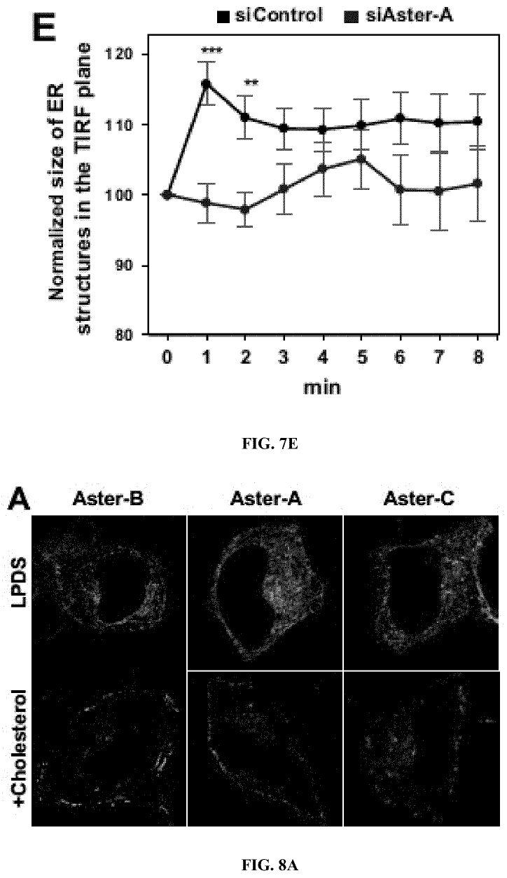

These findings suggested that Aster proteins were facilitating the formation of cholesterol-dependent PM-ER appositions by bridging the two membranes. To better define the nature of these Aster-dependent PM-ER contacts, their relationship to the previously-described PM-ER contacts associated with the proteins ORP5, E-Syt2 and E-Syt3 was assessed. A431 cell lines expressing both BFP-KDEL and Cherry-ORP5, Cherry-E-Syt2, or Cherry-E-Syt3 were generated. The cellular location of GFP-Aster-B in the presence or absence added cholesterol were analyzed. In cells cultured in low-cholesterol media, ORP5, E-Syt2 and E-Syt3 were found predominantly in ER tubules located in proximity to the PM (FIG. 7A, FIG. 7B and FIG. 8D). By contrast, Aster-B was located throughout the ER under these conditions and showed minimal colocalization with ORP5, E-Syt2 or E-Syt3. Addition of cholesterol to the cells had little if any effect on the location of ORP5, E-Syt2 or E-Syt3, but caused a dramatic relocalization of Aster-B to ER tubules that were in close proximity to the PM. Moreover, there was substantial, but not complete, overlap of Aster-B signal with signals for ORP5, E-Syt2 and E-Syt3 in cholesterol-loaded cells. Interestingly, while ER-PM contacts containing Aster-B were frequently located in the same ER tubules in which ORP5, E-Syt2 or E-Syt3 resided, domains containing only Aster-B could also be readily be identified in proximity to the plasma membrane. The colocalization of Aster-B with other PM-ER contact proteins is quantified in FIG. 7C.

Cholesterol-dependent Aster recruitment to the plasma membrane using total internal reflection (TIRF) microscopy, which permits signal detection within 100 nm of the plasma membrane was further analyzed. A431 cells expressing GFP-Aster-B and Cherry-ORP5 after loading of the cells with cyclodextrin cholesterol were analyzed. While ORP5 was detected in the TIRF plane regardless of cellular sterol status, Aster-B was rapidly recruited to the TIRF plane in response to cholesterol (FIG. 7D). These studies conclude that, in contrast to ORP5, E-Syt2 and E-Syt3, which reside in ER-PM contact sites regardless of cellular sterol status, Aster-B is selectively recruited to form distinct ER-PM contacts in response to excess cholesterol in the PM.

The influence of cholesterol loading on the size of ER structures in close proximity the PM was assessed by TIRF video microscopy. U2OS cells stably expressing Cherry-KDEL were treated with control or Aster-A-specific siRNA. Video imaging was then performed to assess the size of ER foci in the TIRF plane following cholesterol loading. Cholesterol administration resulted in the enlargement of ER-structures in close proximity to the plasma membrane and this effect was dependent on Aster-A expression (FIG. 7E).

### Example 5: The Gram Domain Mediates Cholesterol-Dependent Aster Localization at the Plasma Membrane

### Phospholipid Binding Assays

Lipid binding analysis of 6xHis-tagged (“6xHis-tagged” disclosed as SEQ ID NO: 11) B GRAM (Aster-B₁₋₃₃₇) was conducted using PIP Strips (Echelon Biosciences), with each spot containing 100 pmol of active lipids. Membranes were blocked with PBS Tween (PB ST) solution (supplemented with 3% fatty acid free BSA) for 1 hr at room temperature, and incubated with B GRAM fusion protein in blocking buffer for 1 hr. After three washes, the membranes were blotted with anti-His antibody (Biorad, MCA1396GA). The strip contained 15 different types of lipids. LPA, lysophosphatidic acid; LPC, lysophosphatidylcholine; PI, phosphatidylinositol; PI(3)P, phosphatidylinositol 3-phosphate; PI(4)P, phosphatidylinositol 4-phosphate; PI(5)P, phosphatidylinositol 5-phosphate; PE, phosphatidylethanolamine; PC, phosphatidylcholine; S1P, sphingosine-1-phosphate; PI(3,4)P2, phosphatidylinositol 3,4-phosphate; PI(3,5)P2, phosphatidylinositol 3,5-phosphate; PI(4,5)P2, phosphatidy-linositol 4,5-phosphate; PI(3,4,5)P3, phosphatidylinositol 3,4,5-phosphate; PA, phosphatidic acid; PS, phosphatidylserine. Results were confirmed with a FLAG tagged B GRAM Fusion construct.

The Aster proteins contain an N-terminal GRAM domain, a structural motif related to the pleckstrin homology domain that is found in glucosyltransferases, Rab-like GTPase activators, myotubularins, and other membrane-associated proteins. Interestingly, the GRAM domains of myotubularins have been shown to interact with phospholipids. The purified Aster-B GRAM domain interacted strongly with both phosphatidylserine (PS) and phosphatidic acid (PA) (FIG. 9A). Given that phosphatidylserine is highly enriched in the inner leaflet of the PM, it was further determined if the Aster-B GRAM domain was associated with phosphatidylserine-containing liposomes. Co-sedimentation assays revealed that the GRAM domain pelleted with phosphatidylserine-containing, but not phosphatidylcholine-containing, liposomes (FIG. 9B). The inclusion of cholesterol in PS-containing liposomes did not enhance their association with the GRAM domain.

To determine if the Aster GRAM domain is required for the formation of Aster-dependent ER-PM contact sites, Hela and A431 cells were transfected with mutant form of Aster-B in which the GRAM domain had been deleted (FIG. 9C and FIG. 8E). Loss of the GRAM domain abolished recruitment of Asters to the PM in response to cholesterol loading. The Aster-B GRAM domain alone was expressed in CHO-K1 and A431 cells (FIG. 9D). Under basal culture conditions, the soluble GRAM domain was cytoplasmic, but it was recruited to the PM upon cholesterol loading. TIRF microscopy confirmed that C-terminal GFP-tagged Aster-B GRAM domain was largely cytoplasmic in cells cultured in medium containing lipoprotein-deficient serum (LPDS) but that it was recruited to the PM in a time- and concentration-dependent manner after cholesterol loading (FIG. 9E). These studies conclude that the phosphatidylserine-binding GRAM domain is both necessary and sufficient for cholesterol-dependent Aster redistribution to the PM.

### Example 6: Aster-B is Required for Adrenal Sterol Homeostasis Mice

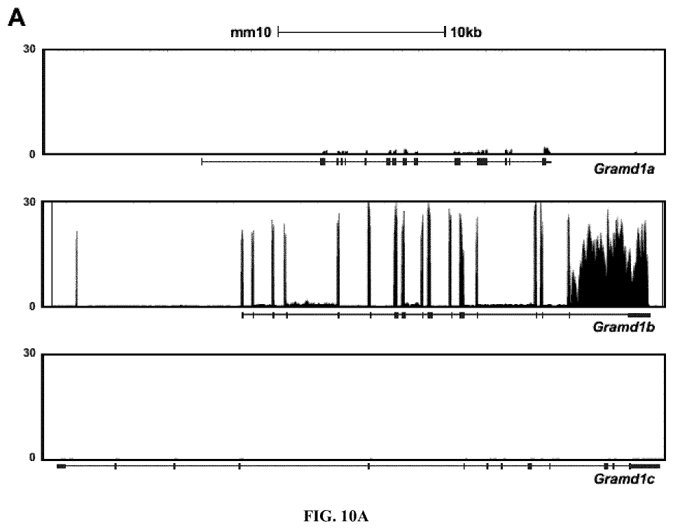

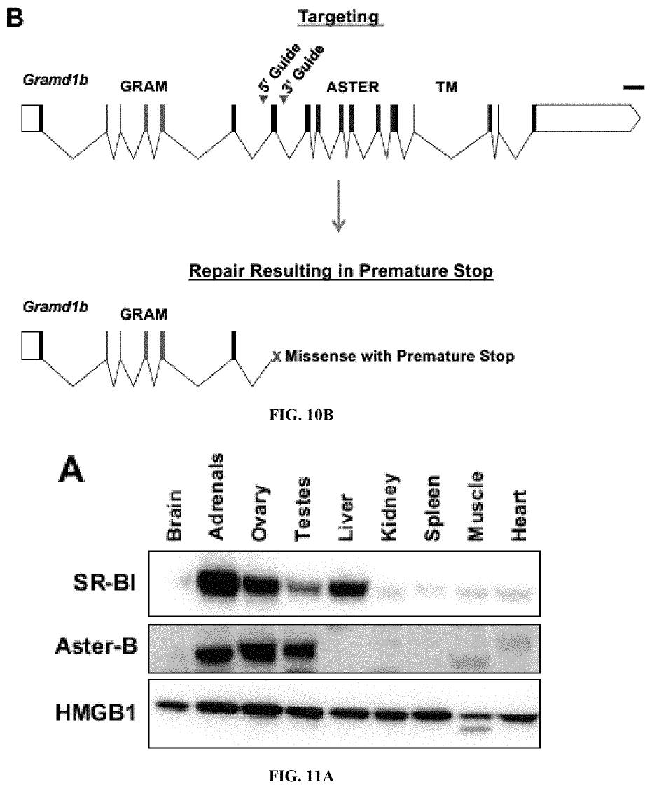

All mice were housed in a temperature-controlled room under a 12 hr light-dark cycle and under pathogen-free conditions. Mice were placed on a chow diet. Experiments were performed in male and female mice. Aster-B global knockout mice were generated at Mouse Biology Program facility on a C57BL/6N background using the CRISPR/Cas9 strategy outlined in FIG. 10B. Experimental mice were sacrificed at ages 6-12 weeks unless otherwise specified for histological, serum, lipid, and gene expression analyses.

### Protein Analysis

Whole cell lysate or tissue lysate was extracted using RIPA lysis buffer (Boston Bioproducts) supplemented with complete protease inhibitor cocktail (Roche). Proteins were diluted in Nupage loading dye (Invitrogen), heated at 95° C. for 5 min, and run on 4-12% NuPAGE Bis-Tris Gel (Invitrogen). Proteins were transferred to hybond ECL membrane (GE Healthcare), blocked with 5% milk (or 5% BSA for anti-SREBP-2) to quench nonspecific protein binding and blotted with the indicated primary antibody. Horseradish peroxidase-conjugated anti-mouse, anti-goat and anti-rabbit IgG (Jackson) were used as secondary antibodies. The immune signal was visualized using the ECL kit (Amersham Biosciences). Nuclei from mouse adrenal glands were prepared by douncing tissue with a motorized overhead stirrer (Caframo Model BDC2002) in 10 mM Hepes-KOH, pH 7.4, 10 mM KCl, 1.5 mM MgCl2, 0.5 mM EDTA sodium, 0.5 mM EGTA sodium, 1 mM DTT, and protease inhibitors. Samples were centrifuged at 1,000×g at 4° C. for 5 minutes to isolate nuclei. Nuclei were washed and resuspended in 10 mM Hepes-KOH, pH 7.4, 0.42 M NaCl, 2.5% glycerol (w/v), 1.5 mM MgCl2, 0.5 mM EDTA sodium, 0.5 mM EGTA sodium, 1 mM DTT, and protease inhibitors. Nuclei were then incubated on ice for 40 minutes with intermittent pipetting, then centrifuged at 10,000×g at 4° C. for 10 minutes. The supernatant was then used as nuclear protein extract. The 1,000×g supernatant was used to prepare a membrane lysate by centrifugation at 100,000×g at 4° C. for 30 minutes, followed by resuspension in RIPA buffer.

### Oil Red O Staining

Oil Red O staining was performed as described (Mehlem et al., 2013). Adrenal glands were dissected carefully and the surrounding fat tissue was removed. After the collected glands were embedded into Tissue-Tek O.C.T. compound (cat No. 4583), placed on dry ice for twenty minutes, then moved to −80° C. Tissue was sectioned (12 μm thick) using a Microm HM 505 E cryostat and sectioned were placed on glass microscope slides (Superfrost plus). Before staining, sections were allowed to equilibrate to room temperature for 10 minutes. Oil Red O solution working solution (Sigma, cat. No. 00625, ˜0.4%) was freshly prepared and filtered before covering the sections. Sections were incubated for 10 minutes, then washed under running tap water for 30 minutes. Sections were then mounted on slides with water-soluble mounting medium (Sigma cat. No. GG1) and images were captured on a Zeiss Axioskop 2 plus bright-field light microscope at a ×40 magnification. Background was corrected by white balance, selected as a blank area outside the section.

### Electron Microscopy

A431 cell monolayers were treated as indicated, then fixed for 1 h in fixative solution containing 4% paraformaldehyde (EMS; Hatfield, Pa.) 2.5% glutaraldehyde (EMS; Hatfield, Pa.) buffered with 0.1M sodium cacodylate (Sigma; Burlington, Mass.). Next, the cells were gently scraped from the dishes using a cell scraper (Corning; Corning, N.Y.). The suspension was then centrifuged at 350×g for 15 min to generate a pellet. The pellets were then allowed to fix for another 45 min. Next, pellets were rinsed 3 times with 0.1M sodium cacodylate before being post-fixed with 1% osmium tetroxide (EMS; Hatfield, Pa.), 1.2% potassium ferricyanide (EMD; Darmstadt, Germany) buffered with 0.1M sodium cacodylate for 1 h at room temperature. Next, samples were rinsed 3 times with distilled H₂O and stained overnight with 2% uranyl acetate at 4° C. Next day, samples were rinsed three times with distilled H₂O and dehydrated through a series of increasing acetone concentrations (30,50, 70, 85, 95, 100%×3, 10 min each) before being infiltrated with increasing concentrations of EMBed812 epoxy resin (EMS; Hatfield, Pa.) in acetone (33% 2 h, 66% overnight, 100% 4 h). Next, samples were embedded in fresh resin and polymerized in a vacuum oven for 24 h at 65° C. The polymerized blocks were removed from the tubes, trimmed and 65 nm sections were made with a Leica UC6 ultramicrotome and picked up on freshly glow-discharged copper grids (Ted Pella; Redding, Calif.) that were coated with formvar and carbon. Sections on grids were then stained with Reynold's lead citrate solution for 10 min. Images were acquired with an FEI T12 transmission electron microscope set to 120 kV accelerating voltage using a Gatan 2kX2k digital camera.

Mice were perfused with 0.1 M Sodium Cacodylate buffer (pH 7.4) and fixed with cold 1.5% Glutaraldehyde, 4% PVP, 0.05% CaCl₂) in 0.1 M Sodium Cacodylate buffer (pH 7.4). Standard transmission EM ultrastructural analysis was performed on adrenal glands with imidazole staining and visualized with a JEOL JEM-123 40-120 kV transmission electron microscope at the Gladstone Electron Microscopy Core.

### Lipid Analysis

Adrenal glands were weighed and snap-frozen in liquid nitrogen. Blood was centrifuged and serum was snap frozen. 10 μl serum was used for analysis. Adrenal glands were pulverized using a hand-held pestle grinder. A modified Bligh-Dyer lipid extraction, in the presence of lipid class internal standards including [25,26,26,26-d₄]-cholesterol and cholesteryl heptadecanoate, was performed on 5-10 mg of pulverized tissue. Lipid extracts were dried under nitrogen and diluted in chloroform/methanol (2/1, v/v). Molecular species were quantified using ESI/MS on a triple-quadrupole instrument (Thermo Fisher Quantum Ultra) utilizing shotgun lipidomics methodologies. Free cholesterol was first derivatized with acetyl chloride and then quantified in positive ion mode using product ion scanning for 83.03 amu (collision energy=18 eV). CE molecular species were quantified using neutral loss scanning for 368.5 amu (collision energy=25 eV). Individual molecular species were calculated by comparing the ion intensities of the molecular species to the ion intensity of the lipid class internal standard as previously described. Serum corticosterone was measured by ELISA kit following cardiac puncture per manufacturer's instructions (Cayman Chemical, cat No. 501320).

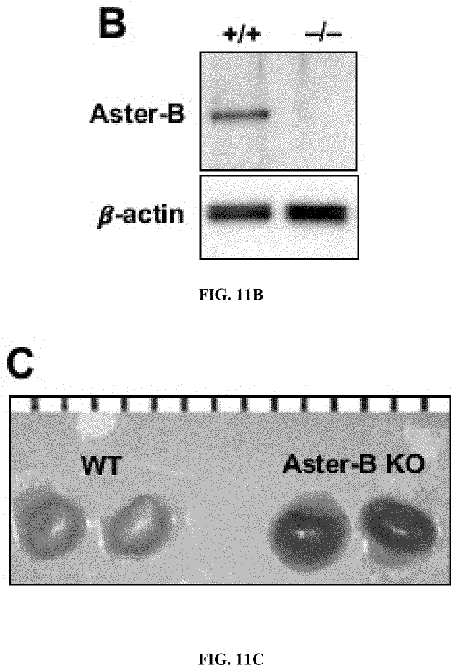

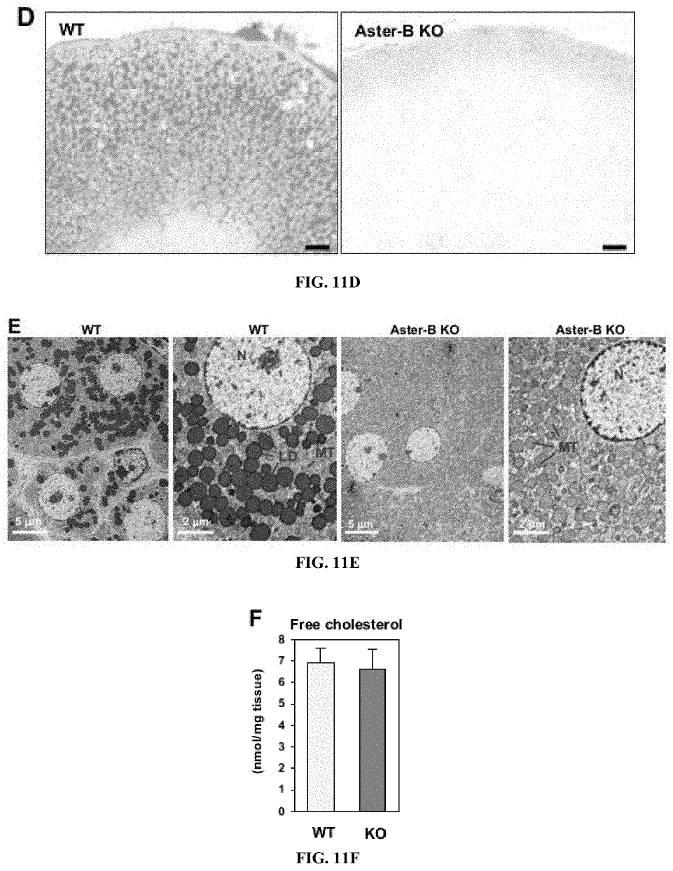

Movement of cholesterol from the PM to the ER is believed to be an important step in the utilization of HDL-derived cholesterol after selective uptake by SR-BI. To confirm that Asters contributes to cholesterol transport from SR-BI at the PM to the ER, the rodent adrenal gland, which depends on SR-BI-mediated uptake of HDL cholesterol for steroidogenesis, was utilized. Among the Aster proteins, Aster-B exhibited the highest expression in the adrenal gland, suggesting that it was likely to be the most physiologically relevant Aster family member in this tissue (FIG. 10A). Using an antibody that was generated against Aster-B, it was confirmed that Aster-B is expressed at high levels in the adrenal, similar to SR-BI (FIG. 11A).

To assess the physiological relevance of cholesterol transport by Aster-B, knockout mice were generated by CRISPR/Cas9 genome editing. exon 7 was deleted resulting in a frameshift mutation (FIG. 10B). The absence of Aster-B was confirmed in homozygous Aster-B knockout mice (FIG. 11B). By visual inspection, the adrenal glands of Aster-B-deficient mice were red (rather than a pinkish white), suggesting an absence of cholesterol ester stores (FIG. 11C). Indeed, oil red 0 staining revealed a complete loss of neutral lipid stores in the adrenal cortex in Aster-B-deficient mice (FIG. 11D), and electron microscopy revealed a complete absence of cytosolic lipid droplets (FIG. 11E). Analysis of tissue lipids showed that, while levels of free cholesterol in the adrenal gland were not different between genotypes, loss of Aster-B expression led to a dramatic decrease in cholesterol esters (FIG. 11F, and FIG. 11G). These findings show that loss of Aster-B expression abolishes tissue cholesterol homeostasis in vivo.

### Example 7: Aster-B Promotes PM to ER Cholesterol Transport

### In Vitro Antisense Oligonucleotide (ASO) Studies

Generation 2.5 constrained ethyl ASOs were synthesized as described previously (Seth et al., 2009). For in vitro knockdown studies, control or Aster-A ASO (GTGGAATTTATTCAGG (SEQ ID NO: 13)) was used. Undifferentiated murine 3T3-L1 cells were plated in 10% FBS DMEM on Day 0. On Day 1 cells were washed and supplemented with 1% LPDS. Cells were then transfected with ASOs using Dharmafect 1 reagent per the manufacturer's recommendations for knockdown studies (Dharmacon, 50 nM final concentration). On Day 2, the medium was changed to fresh DMEM with 1% LPDS, simvastatin (5 μM) and mevalonate (50 μM). On Day 3 cells were treated as described in FIG. 7A and samples were collected. The rate of incorporation of [³H]oleate into cholesteryl [³H]oleate was performed on Day 3 as previously described (Goldstein et al., 1983).

### Lipoprotein Fractionation

Purification of HDL2 particles was performed using potassium bromide density centrifugation from pooled samples of human plasma obtained from the Finnish Red Cross as described previously (Nguyen et al., 2012). Lipid content of HDL2 and LDL fractions was quantified using lipid extraction and thin layer chromatography as described before (Bautista et al., 2014).

The consequence of loss of Aster function for sterol movement from the PM to the ER in cultured cells was investigated. Since Aster-B expression is minimal in most cultured cell lines, knockdown Aster-A expression, which is abundant 3T3-L1 cells, was chosen. When ER cholesterol levels rise, cells respond first by suppressing SREBP-2 cleavage (thereby blocking sterol synthesis) and second by activating ACAT-dependent CE synthesis. Cholesterol movement to the ER following exogenous delivery was assessed by measuring two endpoints: 1. the activity of the SREBP-2 pathway and 2. the formation of cholesterol esters. Treatment of 3T3-L1 cells with an Aster-A-specific ASO (which nearly abolished Gramd1a expression; FIG. 13) led to induction of SREBP-2 processing, increased LDLR protein levels, and increased expression of SREBP-2 target genes, including Hmcgr, and Hmgcs (FIG. 13A and FIG. 13B). Such a change SREBP-2 processing is demonstrative of a change in ER cholesterol levels. Moreover, the ability of exogenously added cyclodextrin-cholesterol to suppress SREBP-2 processing was clearly delayed in Aster-A silenced cells. The fact that some suppression of the SREBP-2 pathway at later time points was still observed even with Aster-A ASO was not unexpected, since vesicular sterol transport pathways were presumably still operative. Interestingly, expression of Abca1, which is controlled by the sterol-activated nuclear receptor LXR, was reciprocally reduced in the absence of Aster-A, both at the protein (FIG. 13A) and the mRNA level (FIG. 13C), consistent with reduced intracellular cholesterol availability.

To assess the effects of Aster deficiency on CE production, cells were incubated in the presence of [³H]oleate, treated with cyclodextrin-cholesterol complexes, and the incorporation of label into CE was quantified. The rate at which cholesterol delivered to the PM, and was incorporated into CE was markedly slower in cells in which Aster-A was silenced (FIG. 13D). Two hours after cholesterol addition the amount of CE formed in Aster-A-silenced cells was less than 25% of controls cells. Again, the fact that some CE could still be formed in the absence of Aster-A was not unexpected given that vesicular transport pathways were intact.

Rodent adrenal glands rely on the selective uptake of HDL-cholesterol by SR-BI to provide free cholesterol for the generation of cholesterol esters by the ER enzyme ACAT1. Interestingly, the adrenal phenotype of Aster-B knockout mice is virtually identical to that described for mice lacking SR-BI or ACAT1. To further address whether Aster-B plays an important role in facilitating the transport of HDL cholesterol, whether HDL-mediated cholesterol delivery to cells affects Aster-B localization was investigated. Both Aster-B and SR-BI were expressed in A431 cells and incubated them with HDL (FIG. 12A, and FIG. 12B). TIRF microscopy showed that incubation of cells with HDL2 stimulated recruitment of Aster-B to the PM (FIG. 12C-FIG. 12E).

Next whether Aster-B was important for cholesterol delivery to adrenal cortical ER in vivo was assessed. A failure to transport HDL-cholesterol from SR-BI at the PM to the ER in adrenocortical cells would be expected to reduce levels of cholesterol in the ER and render the cells dependent on endogenous cholesterol synthesis for production of cortisol. To test whether the ER in Aster-B adrenal cortex was deficient in cholesterol, the expression of SREBP-2 target genes, whose expression is tightly linked to the cholesterol content of ER membranes, was analyzed. SREBP-2 target gene expressing in the adrenal was far higher in Aster-B-deficient mice than in WT mice (FIG. 13E), indicating that the ER is starved for cholesterol in the absence of Aster-B. To confirm that Aster-B deficiency promotes SREBP-2 processing in mice, we prepared nuclear fractions from the adrenal in WT and Aster-B knockout mice were prepared. The nuclear fractions from Aster-B-deficient adrenal glands had dramatically increased levels of the mature SREBP-2, despite comparable levels of the membrane-bound SREBP-2 precursor (FIG. 13F). Normal to elevated levels of SR-BI were observed in Aster-B-deficient mice, indicating that the phenotype was not an indirect consequence of SR-BI deficiency (FIG. 13F).

Cholesterol esters are utilized, particularly during times of stress, for generating corticosteroids. To assess the physiologic consequence of Aster-B deficiency for steroidogenesis, both serum cholesterol and corticosterone levels were assessed. While serum cholesterol levels did not differ significantly between WT and Aster-B knockout mice (FIG. 13G), basal serum corticosterone levels were lower in Aster-B knockout mice than in controls (FIG. 13H). The induction of stress through an overnight fast exacerbated the deficiency in corticosterone. By contrast, levels of epinephrine and dopamine (non-steroid mediators made by the adrenal medulla) were not different between groups (FIG. 12F).

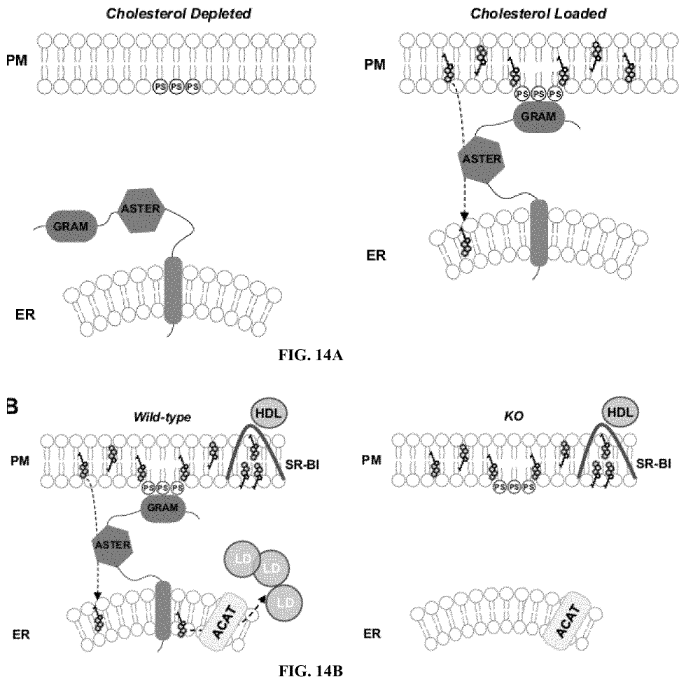

Collectively, the data demonstrate that Aster-B moves cholesterol from the PM to the ER downstream of the HDL receptor SR-BI (FIG. 14). Aster-B is the only mammalian PM-ER cholesterol transporter to show a loss-of-function phenotype in vivo and to be implicated in the transport of HDL-derived cholesterol.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of diagnosing and treating a subject having a condition associated with high cholesterol or at risk for developing a condition associated with high cholesterol, comprising:
a. optionally obtaining a sample from the subject;
b. determining whether the sample from the subject has a decreased Aster protein level or activity compared to a reference level representative of a subject without the condition;
c. if the sample has a decreased Aster protein level or activity compared to the reference level, identifying the subject as having a condition associated with high cholesterol or at risk for developing a condition associated with high cholesterol; and
d. administering a cholesterol-lowering drug to the subject, instructing the subject to reduce cholesterol uptake from diet, and/or administering a reverse cholesterol transport activator.

2-3. (canceled)

4. The method of claim 1, wherein the reference level is the Aster protein level or activity in a normal patient.

5. The method of claim 1, wherein the condition associated with high cholesterol is coronary heart disease, stroke, peripheral vascular disease, erectile dysfunction, diabetes, high blood pressure, cellular cholesterol overload disease, and/or a disease related to defects in brain cholesterol metabolism.

6. The method of claim 5, wherein the cellular cholesterol overload disease is Niemann-Pick type C disease.

7. The method of claim 5, wherein the disease related to defects in brain cholesterol metabolism is Alzheimer's disease (AD), Huntington's disease (HD), or Parkinson's disease (PD).

8. The method of claim 1, wherein the cholesterol-lowering drug is atorvastatin, fluvastatin, lovastatin, pitavastatin, pravastatin, rosuvastatin, simvastatin, niacin, colestipol, cholestyramine, colesevelam, ezetimibe, fenofibrate, gemfibrozil, alirocumab, and/or evolocumab.

9. The method of claim 1, wherein the cholesterol-lowering drug is an agent that increases the level or activity of Aster.

10. The method of claim 9, wherein the agent activates the Aster promoter.

11. The method of claim 9, wherein the agent comprises an Aster polypeptide.

12. The method of claim 9, wherein the agent comprises an Aster polynucleotide.

13. The method of claim 1, wherein the Aster is Aster-A, Aster-B, and/or Aster-C.

14. The method of claim 1, wherein the reverse cholesterol transport activator is a liver X receptor (LXR) agonist, an activator of hepatic apoA-I, an inhibitor of cholesteryl ester transfer protein (CETP), or an inhibitor of endothelial lipase (EL).

15. The method of claim 14, wherein the LXR agonist is LXR-623.

16. The method of claim 14, wherein the activator of hepatic apoA-I is RVX-208, apoA-I mimetic peptides or a peroxisome proliferator-activated receptor α (PPARα) agonist.

17. The method of claim 16, wherein the PPARα agonist is LY518764.

18. The method of claim 14, wherein the CETP inhibitor is torcetrapib, anacetrapib, or delcetrapib.

19. The method of claim 14, wherein the EL inhibitor is GSK 264220A.

20. A method of diagnosing and treating a subject having a condition associated with high cholesterol or at risk for developing a condition associated with high cholesterol, comprising:
a. optionally obtaining a sample from the subject;
b. analyzing the sample to detect the presence of one or more mutant Aster polynucleotide molecules, and/or one or more mutant Aster polypeptides;
c. if the subject has one or more mutant Aster polynucleotide molecules, and/or one or more mutant Aster polypeptides, identifying the subject as having a condition associated with high cholesterol or at risk for developing a condition associated with high cholesterol;
d. administering a cholesterol-lowering drug to the patient, instructing the subject to reduce cholesterol uptake from diet, and/or administering a reverse cholesterol transport activator.

21-36. (canceled)

37. A method of treating a subject having a condition associated with high cholesterol or at risk for developing a condition associated with high cholesterol, comprising administering to the subject an agent that increases the level or activity of Aster.

38-63. (canceled)


SEQUENCE LISTING (20 sequences)

SEQ ID NO: 1 - protein, 724 aa, Homo sapiens
MFDTTPHSGRSTPSSSPSLRKRLQLLPPSRPPPEPEPGTMVEKGSDSSSEKGGVPGTPSTQSLGSRNFIRNSKKMQSWYSMLSPTYKQRNEDFRKLFSKLPEAERLIVDYSCALQREILLQGRLYLSENWICFYSNIFRWETTISIQLKEVTCLKKEKTAKLIPNAIQICTESEKHFFTSFGARDRCFLLIFRLWQNALLEKTLSPRELWHLVHQCYGSELGLTSEDEDYVSPLQLNGLGTPKEVGDVIALSDITSSGAADRSQEPSPVGSRRGHVTPNLSRASSDADHGAEEDKEEQVDSQPDASSSQTVTPVAEPPSTEPTQPDGPTTLGPLDLLPSEELLTDTSNSSSSTGEEADLAALLPDLSGRLLINSVFHVGAERLQQMLFSDSPFLQGFLQQCKFTDVTLSPWSGDSKCHQRRVLTYTIPISNPLGPKSASVVETQTLFRRGPQAGGCVVDSEVLTQGIPYQDYFYTAHRYCILGLARNKARLRVSSEIRYRKQPWSLVKSLIEKNSWSGIEDYFHHLERELAKAEKLSLEEGGKDARGLLSGLRRRKRPLSWRAHGDGPQHPDPDPCARAGIHTSGSLSSRFSEPSVDQGPGAGIPSALVLISIVICVSLIILIALNVLLFYRLWSLERTAHTFESWHSLALAKGKFPQTATEWAEILALQKQFHSVEVHKWRQILRASVELLDEMKFSLEKLHQGITVSDPPFDTQPRPDDSFS

SEQ ID NO: 2 - protein, 698 aa, Homo sapiens
MVEKGSDHSSDKSPSTPEQGVQRSCSSQSGRSGGKNSKKSQSWYNVLSPTYKQRNEDFRKLFKQLPDTERLIVDYSCALQRDILLQGRLYLSENWICFYSNIFRWETLLTVRLKDICSMTKEKTARLIPNAIQVCTDSEKHFFTSFGARDRTYMMMFRLWQNALLEKPLCPKELWHFVHQCYGNELGLTSDDEDYVPPDDDFNTMGYCEEIPVEENEVNDSSSKSSIETKPDASPQLPKKSITNSTLTSTGSSEAPVSFDGLPLEEEALEGDGSLEKELAIDNIMGEKIEMIAPVNSPSLDFNDNEDIPTELSDSSDTHDEGEVQAFYEDLSGRQYVNEVFNFSVDKLYDLLFTNSPFQRDFMEQRRFSDIIFHPWKKEENGNQSRVILYTITLTNPLAPKTATVRETQTMYKASQESECYVIDAEVLTHDVPYHDYFYTINRYTLTRVARNKSRLRVSTELRYRKQPWGLVKTFIEKNFWSGLEDYFRHLESELAKTESTYLAEMHRQSPKEKASKTTTVRRRKRPHAHLRVPHLEEVMSPVTTPTDEDVGHRIKHVAGSTQTRHIPEDTPNGFHLQSVSKLLLVISCVICFSLVLLVILNMMLFYKLWMLEYTTQTLTAWQGLRLQERLPQSQTEWAQLLESQQKYHDTELQKWREIIKSSVMLLDQMKDSLINLQNGIRSRDYTSESEEKRNRYH

SEQ ID NO: 3 - protein, 662 aa, Homo sapiens
MEGAPTVRQVMNEGDSSLATDLQEDVEENPSPTVEENNVVVKKQGPNLHNWSGDWSFWISSSTYKDRNEEYRRQFTHLPDTERLIADYACALQRDILLQGRLYLSENWLCFYSNIFRWETTISIALKNITFMTKEKTARLIPNAIQIVTESEKFFFTSFGARDRSYLSIFRLWQNVLLDKSLTRQEFWQLLQQNYGTELGLNAEEMENLSLSIEDVQPRSPGRSSLDDSGERDEKLSKSISFTSESISRVSETESFDGNSSKGGLGKEESQNEKQTKKSLLPTLEKKLTRVPSKSLDLNKNEYLSLDKSSTSDSVDEENVPEKDLHGRLFINRIFHISADRMFELLFTSSRFMQKFASSRNIIDVVSTPWTAELGGDQLRTMTYTIVLNSPLTGKCTAATEKQTLYKESREARFYLVDSEVLTHDVPYHDYFYTVNRYCIIRSSKQKCRLRVSTDLKYRKQPWGLVKSLIEKNSWSSLEDYFKQLESDLLIEESVLNQAIEDPGKLTGLRRRRRTFNRTAETVPKLSSQHSSGDVGLGAKGDITGKKKEMENYNVTLIVVMSIFVLLLVLLNVTLFLKLSKIEHAAQSFYRLRLQEEKSLNLASDMVSRAETIQKNKDQAHRLKGVLRDSIVMLEQLKSSLIMLQKTFDLLNKNKTGMAVES

SEQ ID NO: 4 - protein, 722 aa, Mus musculus
MFDTTPHSGRSSPSSSPSLRKRLQLLPPIRPPPASEPEPGTMVEKGSDSSSEKSGVSGTLSTQSLGSRNFIRNSKKMQSWYSMLCPTYKQRNEDFRKLFSKLPEAERLIVDYSCALQREILLQGRLYLSENWICFYSNIFRWETTISIQLKEVTCLKKEKTAKLIPNAIQICTESEKHFFTSFGARDRCFLLIFRLWQNALLEKTLSPRELWHLVHQCYGSELGLTSEDEDYVCPLQLNGLGSPKEVGDVIALSDISPSGAADHSQEPSPVGSRRGRVTPNLSRASSDADHGAEEDKEEQTDGLDASSSQTVTPVAEPLSSEPTPPDGPTSSLGPLDLLSREELLTDTSNSSSSTGEEGDLAALLPDLSGRLLINSVFHMGAERLQQMLFSDSPFLQGFLQQRKFTDVTLSPWSSDSKCHQRRVLTYTIPISNQLGPKSASVVETQTLFRRGPQAGGCVVDSEVLTQGIPYQDYFYTAHRYCILGLARNKARLRVSSEIRYRKQPWSLVKSLIEKNSWSGIEDYFHHLDRELAKAEKLSLEEGGKDTRGLLSGLRRRKRPLSWRGHRDGPQHPDPDPCTQTSMHTSGSLSSRFSEPSVDQGPGAGIPSALVLISIVLIVLIALNALLFYRLWSLERTAHTFESWHSLALAKGKFPQTATEWAEILALQKHFHSVEVHKWRQILRASVELLDEMKFSLEKLHQGITVPDPPLDTQPQPDDSFP

SEQ ID NO: 5 - protein, 698 aa, Mus musculus
MESLTESGVLWSLLLELDSQSLLWYLKRLADAPVGAECYCWHGSEKIPAVLSPTYKQRNEDFRKLFKQLPDTERLIVDYSCALQRDILLQGRLYLSENWICFYSNIFRWETLLTVRLKDICSMTKEKTARLIPNAIQVCTDSEKHFFTSFGARDRTYMMMFRLWQNALLEKPLCPKELWHFVHQCYGNELGLTSDDEDYVPPDDDFNTMGYCEEIPIEENEVNDSSSKSSIETKPDASPQLPKKSITNSTLTSTGSSEAPVSFDGLPLEEEVMEGDGSLEKELAIDNIIGEKIEIMAPVTSPSLDFNDNEDIPTELSDSSDTHDEGEVQAFYEDLSGRQYVNEVFNFSVDKLYDLLFTNSPFLRDFMEQRRFSDIIFHPWKKEENGNQSRVILYTITLTNPLAPKTATVRETQTMYKASQESECYVIDAEVLTHDVPYHDYFYTINRYTLTRVARNKSRLRVSTELRYRKQPWGFVKTFIEKNFWSGLEDYFRHLETELTKTESTYLAEIHRQSPKEKASKSSAVRRRKRPHAHLRVPHLEEVMSPVTTPTDEDVGHRIKHVAGSTQTRHIPEDTPDGFHLQSVSKLLLVISCVLVLLVVLNMMLFYKLWMLEYTTQTLTAWQGLRLQERLPQSQTEWAQLLESQQKYHDTELQKWREIIKSSVLLLDQMKDSLINLQNGIRSRDYTAESDEKRNRYH

SEQ ID NO: 6 - protein, 662 aa, Mus musculus
MEGALTARQIVNEGDSSLATELQEEPEESPGPVVDENIVSAKKQGQSTHNWSGDWSFWISSSTYKDRNEEYRQQFTHLPDSEKLIADYACALQKDILVQGRLYLSEKWLCFYSNIFRWETTISIALKNITFMTKEKTARLIPNAIQIITEGEKFFFTSFGARDRSYLIIFRLWQNVLLDKSLTRQEFWQLLQQNYGTELGLNAEEMEHLLSVEENVQPRSPGRSSVDDAGERDEKFSKAVSFTQESVSRASETEPLDGNSPKRGLGKEDSQSERNVRKSPSLASEKRISRAPSKSLDLNKNEYLSLDKSSTSDSVDEENIPEKDLQGRLYINRVFHISAERMFELLFTSSHFMQRFANSRNIIDVVSTPWTVESGGNQLRTMTYTIVLSNPLTGKYTAATEKQTLYKESREAQFYLVDSEVLTHDVPYHDYFYTLNRYCIVRSAKQRCRLRVSTDLKYRKQPWGLIKSLIEKNSWSSLESYFKKLESDLLMEESVLSQSIEDAGKHSSLRRRRRTLNRTAEPVPKLSSQRSSTDLGFEAKVDVTGKRKTVDSYDTALIVVMSIFLLLLVLLNVTLFLKLSKIEHATQSFYQLHLQGEKSLNLVSDRFSRTENIQKNKDQAHRLKGVLQDSIVMLEQLKSSLIMLQKTFDLLNKNKSGVAVES

SEQ ID NO: 7 - protein, 20 aa, Artificial Sequence
  Description of Artificial Sequence Synthetic peptide
MLCCMRRTKQVEKNDEDQKI

SEQ ID NO: 8 - DNA, 32 bp, Artificial Sequence
  Description of Artificial Sequence Synthetic primer
atctagatctaccatgggctgctccgccaaag

SEQ ID NO: 9 - DNA, 30 bp, Artificial Sequence
  Description of Artificial Sequence Synthetic primer
atttgcggccgcgtgtgtgcaggtgtgcaa

SEQ ID NO: 10 - protein, 20 aa, Artificial Sequence
  Description of Artificial Sequence Synthetic peptide
KLNPPDESGPGCMSCKCVLS

SEQ ID NO: 11 - protein, 6 aa, Artificial Sequence
  Description of Artificial Sequence Synthetic 6xHis tag
HHHHHH

SEQ ID NO: 12 - DNA, 21 bp, Unknown
  Description of Unknown Target sequence
cacgatctccatccagctgaa

SEQ ID NO: 13 - DNA, 16 bp, Unknown
  Description of Unknown Aster-A ASO sequence
gtggaatttattcagg

SEQ ID NO: 14 - protein, 175 aa, Unknown
  Description of Unknown Mammalian Aster sequence
LPDLSGRLLINSVFHMGAERLQQMLFSDSPFLQGFLQQRKFTDVTLSPWSSDSKCHQRRVLTYTIPISNQLGPKSASVVETQTLFRRGPQAGGCVVDSEVLTQGIPYQDYFYTAHRYCILGLARNKARLRVSSEIRYRKQPWSLVKSLIEKNSWSGIEDYFHHLDRELAKAEKLS

SEQ ID NO: 15 - protein, 175 aa, Unknown
  Description of Unknown Mammalian Aster sequence
YEDLSGRQYVNEVFNFSVDKLYDLLFTNSPFLRDFMEQRRFSDIIFHPWKKEENGNQSRVILYTITLTNPLAPKTATVRETQTMYKASQESECYVIDAEVLTHDVPYHDYFYTINRYTLTRVARNKSRLRVSTELRYRKQPWGFVKTFIEKNFWSGLEDYFRHLETELTKTESTY

SEQ ID NO: 16 - protein, 175 aa, Unknown
  Description of Unknown Mammalian Aster sequence
EKDLQGRLYINRVFHISAERMFELLFTSSHFMQRFANSRNIIDVVSTPWTVESGGNQLRTMTYTIVLSNPLTGKYTAATEKQTLYKESREAQFYLVDSEVLTHDVPYHDYFYTLNRYCIVRSAKQRCRLRVSTDLKYRKQPWGLIKSLIEKNSWSSLESYFKKLESDLLMEESVL

SEQ ID NO: 17 - protein, 169 aa, Saccharomyces cerevisiae
KPSNNDHLVIEANINAPLGKVVNLLYGEDVSYYERILKAQKNFEISPIPNNFLTKKIRDYAYTKPLSGSIGPSKTKCLITDTLEHYDLEDYVKVLSITKNPDVPSGNIFSVKTVFLFSWDKNNSTKLTVYNSVDWTGKSWIKSMIEKGTFDGVADTTKIMISEIKKILS

SEQ ID NO: 18 - protein, 170 aa, Saccharomyces cerevisiae
KPAPNEKLVNESTIHASLGRVVNILFGKDVSYIMAILKAQKNSDISPIPVLVDSPTVSEGKKRDYSYVKTTPGAIGPGKTKCMITETIQHFNLEEYVQVLQTTKTPDVPSGNSFYVRTVYLLSWANNNETKLKLYVSVEWTGKSLIKSPIEKGTFDGVTDATKILVEELG

SEQ ID NO: 19 - protein, 169 aa, Saccharomyces cerevisiae
KGKDDTVIDEKINIPVPLGTVFSLLYGDDTSYIKKIIENQNNFNVCDIPKFVNNAREITYTKKLNNSFGPKQTKCIVTETIEHMDLNSFFMVKQIVRSPDVPYGSSFSVHTRFFYSWGDHNTTNMKVVTNVVWTGKSMLKGTIEKGSIDGQRSSTKQLVDDLKKIISNA

SEQ ID NO: 20 - protein, 175 aa, Saccharomyces cerevisiae
KDKDDSIIRENENIPAPLGTVVQLLFGSNTEYMQKVITRDKNNVNVETIPKFTPSLVEGGSRHYEYTKKLNNSIGPKQTKCLLTESIEHMDINNYVLVTQTTKTPDVPSGSNFAVESKIFLFWGQHDTTNMTVITKINWTSKSFLKGAIEKGSVEGQKVSVDYMLSELRDIISRA